SEQUENCE LISTING (3 sequences)

SEQ ID NO: 1 - protein, 227 aa, Homo sapiens
MGSSHHHHHHSQDPNSMTTLTRQDLNFGQVVADVLCEFLEVAVHLILYVREVYPVGIFQKRKKYNVPVQMSCHPELNQYIQDTLHCVKPLLEKNDVEKVVVVILDKEHRPVEKFVFEITQPPLLSISSDSLLSHVEQLLAAFILKISVCDAVLDHNPPGCTFTVLVHTREAATRNMEKIQVIKDFPWILADEQDVHMHDPRLIPLKTMTSDILKMQLYVEERAHKGS

SEQ ID NO: 2 - protein, 8 aa, Homo sapiens
LLAAFILK

SEQ ID NO: 3 - protein, 18 aa, Homo sapiens
LIPLKTMTSDILKMQLYV

(19) United States
(12) Patent Application Publication
(10) Pub. No.: US 2025/0255876 A1
(43) Pub. Date: Aug. 14, 2025

(54) COMPOSITIONS AND METHODS FOR CANCER TREATMENT

(71) Applicant: ARIEL SCIENTIFIC INNOVATIONS LTD., Ariel (IL)

(72) Inventors: Tamar LISTOVSKY, Ariel (IL); Dror TOBI, Ariel (IL)

(21) Appl. No.: 18/707,014

(22) PCT Filed: Nov. 6, 2022
(86) PCT No.: PCT/IL2022/051173

§ 371 Date: May 2, 2024

Related U.S. Application Data
(60) Provisional application No. 63/276,217, filed on Nov. 5, 2021.
(60) Provisional application No. 63/306,245, filed on Feb. 3, 2022.

(51) Int. Cl. A61K 31/53; A61K 31/415; A61K 31/422; A61K 31/433; A61K 33/243; A61P 35/00

(52) U.S. Cl. CPC A61K 31/53; A61K 31/415; A61K 31/422; A61K 31/433; A61K 33/243; A61P 35/00

(57) ABSTRACT

The present invention provides compounds and pharmaceutical compositions comprising thereof. Further, methods for increasing or prolonging the therapeutic efficacy of a chemotherapeutic agent in a subject in need thereof, and methods for treating or preventing development of cancer in a subject in need thereof are also provided.

### CROSS-REFERENCE TO RELATED APPLICATIONS

This application claims the benefit of priority from U.S. Provisional Patent Application No. 63/276,217, filed on Nov. 5, 2021; and from U.S. Provisional Patent Application No. 63/306,245 filed on Feb. 3, 2022. The contents of the above documents are incorporated by reference in their entirety as if fully set forth herein.

### FIELD OF THE INVENTION

The present invention is in the field of cancer therapy.

### BACKGROUND OF THE INVENTION

DNA damaging agents are widely used in oncology to treat both hematological and solid cancers. Some commonly used modalities include ionizing radiation, platinum-based drugs, cyclophosphamide, chlorambucil, and temozolomide.

Platinum-based drugs, and in particular cis-diamminedichloroplatinum(II) (best known as cisplatin), are employed for the treatment of many types of cancer. Cisplatin exerts anticancer effects via multiple mechanisms, one of which involves the generation of DNA lesions followed by the activation of the DNA damage response and the induction of mitochondrial apoptosis. Despite a consistent rate of initial responses, cisplatin treatment often results in the development of chemoresistance, leading to therapeutic failure.

As in some clinical settings cisplatin and DNA damaging agents constitute the major therapeutic option, there is a need for development of effective chemosensitization strategies.

### SUMMARY OF THE INVENTION

The following embodiments and aspects thereof are described and illustrated in conjunction with systems, tools and methods which are meant to be exemplary and illustrative, not limiting in scope.

In one aspect of the invention, there is a pharmaceutical composition comprising a compound, pharmaceutically acceptable salt, isomer or tautomer thereof, wherein the compound is represented by Formula I:

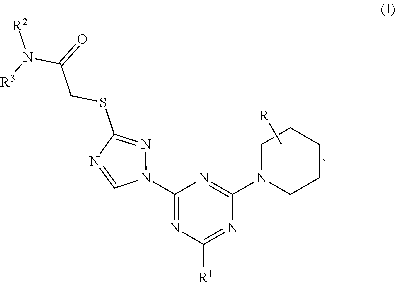

wherein: R, R¹, R² and R³ are independently selected from the group consisting of an optionally substituted heteroaryl, an optionally substituted aryl, a optionally substituted bicyclic aromatic ring, an optionally substituted aliphatic ring, an optionally substituted unsaturated aliphatic ring, an optionally substituted bicyclic aliphatic ring, an optionally substituted linear C₁-C₇ alkyl group, an optionally substituted branched C₁-C₇ alkyl group, an optionally substituted branched C₁-C₇ haloalkyl group, an optionally substituted linear C₁-C₇ haloalkyl group, an optionally substituted C₁-C₇ alkylhydroxy group, an optionally substituted C₁-C₇ alkoxy group, a hydrogen, a heteroatom, a hydroxy group, a halo group, an alkyl group, an alkoxy group, an amino group, or any combination thereof.

In some embodiments, R¹ represents a substituent comprising any one of —NO₂, —CN, —OH, —NH₂, carbonyl, —CONH₂, —CONR₂, —CNNR₂, —CSNR₂, —CONH—OH, —CONH—NH₂, —NHCOR, —NHCSR, —NHCNR, —NC(═O)R, —NC(═O)OR, —NC(═O)NR, —NC(═S)OR, —NC(═S)NR, —SO₂R, —SOR, —SR, —SO₂OR, —SO₂N(R)₂, —NHNR₂, —NNR, C₁-C₇ haloalkyl, optionally substituted C₁-C₁₀ alkyl, —NH(C₁-C₆ alkyl), —N(C₁-C₆ alkyl)₂, C₁-C₆ alkoxy, C₁-C₆haloalkoxy, hydroxy(C₁-C₆ alkyl), hydroxy(C₁-C₆ alkoxy), alkoxy(C₁-C₆ alkyl), alkoxy(C₁-C₆ alkoxy), amino(C₁-C₆ alkyl), —CONH(C₁-C₆ alkyl), —CON(C₁-C₆ alkyl)₂, —CO₂H, —CO₂R, —OCOR, —C(═O)R, —OC(═O)OR, —OC(═O)NR, —OC(═S)OR, —OC(═S)NR, a heteroatom, an optionally substituted cycloalkyl, an optionally substituted heterocyclyl, an optionally substituted aryl, or a combination thereof.

In one aspect of the invention, there is a pharmaceutical composition comprising a compound, pharmaceutically acceptable salt, isomer or tautomer thereof, wherein the compound is represented by Formula II:

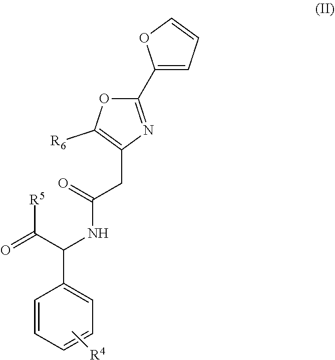

wherein: R⁴, R⁵ and R⁶ are independently selected from the group consisting of a heteroaryl, an aryl, a bicyclic aromatic ring, an aliphatic ring, an unsaturated aliphatic ring, a bicyclic aliphatic ring, a linear C₁-C₇ alkyl group, a branched C₁-C₇ alkyl group, a branched C₁-C₇ haloalkyl group, a linear C₁-C₇ haloalkyl group, a C₁-C₇ alkylhydroxy group, a C₁-C₇ alkoxy group, a hydrogen, a hydroxy group, a halo group, an alkyl group, an alkoxy group, an amino group, or any combination thereof.

In one aspect of the invention, there is a pharmaceutical composition comprising a compound, pharmaceutically acceptable salt, isomer or tautomer thereof, wherein the compound is represented by Formula III:

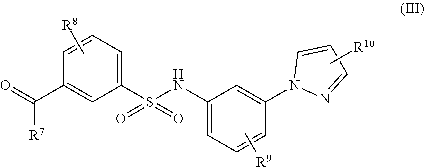

wherein: R⁷, R¹, R⁹ and R¹⁰ are independently selected from the group consisting of a heteroaryl, an aryl, a bicyclic aromatic ring, an aliphatic ring, an unsaturated aliphatic ring, a bicyclic aliphatic ring, a linear C₁-C₇ alkyl group, a branched C₁-C₇ alkyl group, a branched C₁-C₇ haloalkyl group, a linear C₁-C₇ haloalkyl group, a C₁-C₇ alkylhydroxy group, a C₁-C₇ alkoxy group, a hydrogen, a hydroxy group, a halo group, an alkyl group, an alkoxy group, an amino group, or any combination thereof.

In some embodiments, the compound is:

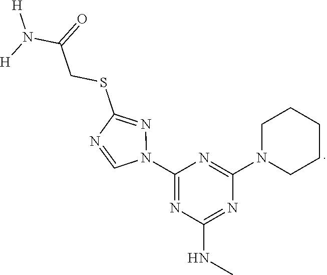 [SMILES: CNc1nc(N2CCCCC2)nc(-n2cnc(SCC(N)=O)n2)n1]

In some embodiments, the compound is:

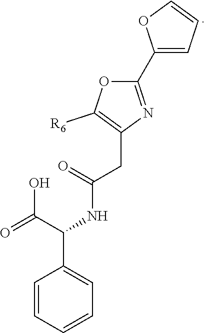

In some embodiments, the compound is:

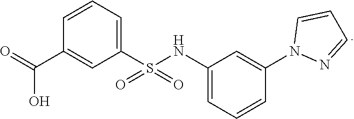 [SMILES: O=C(O)c1cccc(S(=O)(=O)Nc2cccc(-n3cccn3)c2)c1]

In some embodiments, the pharmaceutical composition is for use in combination therapy with a chemotherapeutic agent selected from the group consisting of a crosslinking agent, a strand break agent, an alkylating agent, an anti-metabolite agent, a microtubule disruptor, a radiomimetic agent, a radiosensitizer, an intercalator, a DNA replication inhibitor, an anthracycline, an etoposide, and a topoisomerase II inhibitor.

In some embodiments, the chemotherapeutic agent is selected from the group consisting of cisplatin, carboplatin, etoposide, oxaliplatin, rituximab or trastuzumab, mechlorethamine, cyclophosphamide, bleomycin, doxorubicin, daunorubicin, cytarabine, methotrexate, hydroxyurea, or a combination thereof.

In some embodiments, the compound and the chemotherapeutic agent are present in the composition at a weight to weight (w/w) ratio ranging from 1:0.01 to 0.01:1.

In some embodiments, the pharmaceutical composition further comprises a pharmaceutically acceptable carrier.

In some embodiments, the pharmaceutical composition is for use in the prevention or treatment of a disorder associated with cancer.

In some embodiments, the pharmaceutical composition is for use in increasing the sensitivity of cancer cells to a cancer therapy selected from chemotherapy or radiation therapy.

In some embodiments, the pharmaceutical composition is for use in sensitizing cancer cells, promoting cell death in cancer cells, or inhibiting cell repair from DNA damage.

In one aspect of the invention, there is a method for increasing or prolonging the therapeutic efficacy of a chemotherapeutic agent in a subject in need thereof, comprising administering to the subject a therapeutically effective amount of the pharmaceutical composition of the present invention.

In one aspect of the invention, there is a method for treating or preventing development of cancer in a subject in need thereof, comprising administering to the subject a therapeutically effective amount of (a) chemotherapeutic agent and (b) the pharmaceutical composition of the present invention.

In some embodiments, the cancer is selected from the group consisting of lung cancer, breast cancer, osteosarcoma, neuroblastoma, colon adenocarcinoma, chronic myelogenous leukemia (CML), acute myeloid leukemia (AML), acute promyelocyte leukemia (APL), sarcoma, myxoma, rhabdomyoma, fibroma, lipoma, teratoma; bronchogenic carcinoma, alveolar carcinoma, bronchial adenoma, sarcoma, lymphoma, chondromatous hamartoma, mesothelioma, esophageal cancer, stomach cancer, pancreatic cancer, small bowel cancer, large bowel cancer; kidney cancer, bladder cancer, urethra cancer, prostate cancer, testis cancer; hepatoma, cholangiocarcinoma, hepatoblastoma, angiosarcoma, hepatocellular adenoma, hemangioma, osteogenic sarcoma, fibrosarcoma, malignant fibrous histiocytoma, chondrosarcoma, Ewing's sarcoma, malignant lymphoma, multiple myeloma, malignant giant cell tumor chordoma, osteochronfroma, benign chondroma, chondroblastoma, chondromyxofibroma, osteoid osteoma, giant cell tumors, cancer of the skuli, meninges cancer, brain cancer, spinal cord cancer, uterus cancer, cervical cancer, cancer of the ovaries, vulva cancer, vagina cancer, Hodgkin's disease, non-Hodgkin's lymphoma, malignant melanoma, basal cell carcinoma, squamous cell carcinoma, Karposi's sarcoma, moles dysplastic nevi, lipoma, angioma, and dermatofibroma.

In some embodiments, the chemotherapeutic agent is selected from the group consisting of doxorubicin, cisplatin, oxaliplatin, rituximab, hydroxyurea or trastuzumab.

In one aspect of the invention, there is a kit comprising: (a) the pharmaceutical composition of the present invention; and (b) a chemotherapeutic agent.

In some embodiments, the kit comprises instructions for administering the pharmaceutical composition and the chemotherapeutic agent a w/w ratio ranging from 1:0.01 to 0.01:1.

In one aspect of the invention, there is an agent that binds a region of a Mitotic Arrest Deficient 2 Like 2 protein (MAD2L2; SEQ ID NO: 1; MGSSHHHHHHSQDPNSMTTLTRQDLNFGQVVADVLCEFLEVAVHLILYVREVYP VGIFQKRKKYNVPVQMSCHPELNQYIQDTLHCVKPLLEKNDVEKVVVVILDKEH RPVEKFVFEITQPPLLSISSDSLLSHVEQLLAAFILKISVCDAVLDHNPPGCTFTVLV HTREAATRNMEKIQVIKDFPWILADEQDVHMHDPRLIPLKTMTSDILKMQLYVEE RAHKGS), wherein the region comprises any one of: SEQ ID NO: 2 (LLAAFILK); and SEQ ID NO: 3 (LIPLKTMTSDILKMQLYV).

In some embodiments, the agent inhibits a MAD2L2:Rev1 interaction.

In some embodiments, the agent is for use in increasing the sensitivity of cancer cells to a cancer therapy selected from chemotherapy or radiation therapy.

In some embodiments, the agent is for use in treating or preventing development of cancer.

In some embodiments, the agent is selected from the group consisting of:

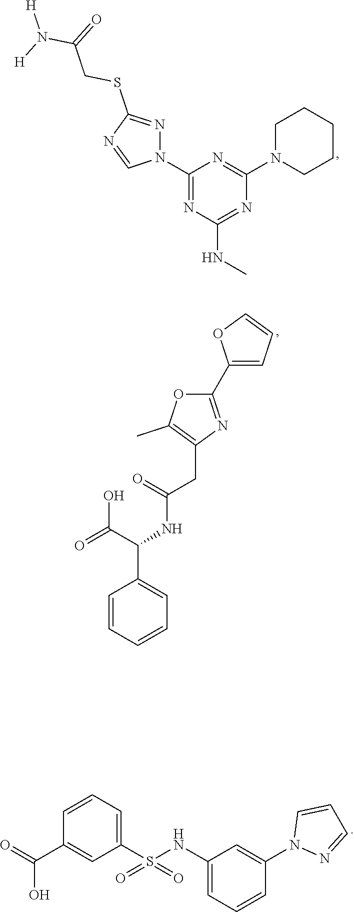

In one aspect of the invention, there is a pharmaceutical composition comprising the agent of the present invention.

In some embodiments, the pharmaceutical composition comprises between 100 nM and 5 mM of the agent.

In some embodiments, the pharmaceutical composition is for use in combination therapy with a chemotherapeutic agent selected from the group consisting of a crosslinking agent, a strand break agent, an alkylating agent, an anti-metabolite agent, a microtubule disruptor, a radiomimetic agent, a radiosensitizer, an intercalator, a DNA replication inhibitor, an anthracycline, an etoposide, and a topoisomerase II inhibitor.

In some embodiments, the chemotherapeutic agent is selected from the group consisting of cisplatin, carboplatin, etoposide, oxaliplatin, rituximab or trastuzumab, mechlorethamine, cyclophosphamide, bleomycin, doxorubicin, daunorubicin, cytarabine, methotrexate, hydroxyurea, or a combination thereof.

In some embodiments, the compound and the chemotherapeutic agent are present in the composition at a weight to weight (w/w) ratio ranging from 1:0.01 to 0.01:1.

In some embodiments, the pharmaceutical composition further comprises a pharmaceutically acceptable carrier.

In some embodiments, the pharmaceutical composition is for use in the prevention or treatment of a disorder associated with cancer.

In some embodiments, the pharmaceutical composition is for use in increasing the sensitivity of cancer cells to a cancer therapy selected from chemotherapy or radiation therapy.

In some embodiments, the pharmaceutical composition is for use in sensitizing cancer cells, promoting cell death in cancer cells, or inhibiting cell repair from DNA damage.

In one aspect of the invention, there is a method for increasing or prolonging the therapeutic efficacy of a chemotherapeutic agent in a subject in need thereof, comprising administering to the subject a therapeutically effective amount of the pharmaceutical composition of the present invention.

In one aspect of the invention, there is a method for treating or preventing development of cancer in a subject in need thereof, comprising administering to the subject a therapeutically effective amount of (a) chemotherapeutic agent and (b) the pharmaceutical composition of the present invention.

In some embodiments, the cancer is selected from the group consisting of lung cancer, breast cancer, osteosarcoma, neuroblastoma, colon adenocarcinoma, chronic myelogenous leukemia (CML), acute myeloid leukemia (AML), acute promyelocyte leukemia (APL), sarcoma, myxoma, rhabdomyoma, fibroma, lipoma, teratoma; bronchogenic carcinoma, alveolar carcinoma, bronchial adenoma, sarcoma, lymphoma, chondromatous hamartoma, mesothelioma, esophageal cancer, stomach cancer, pancreatic cancer, small bowel cancer, large bowel cancer; kidney cancer, bladder cancer, urethra cancer, prostate cancer, testis cancer; hepatoma, cholangiocarcinoma, hepatoblastoma, angiosarcoma, hepatocellular adenoma, hemangioma, osteogenic sarcoma, fibrosarcoma, malignant fibrous histiocytoma, chondrosarcoma, Ewing's sarcoma, malignant lymphoma, multiple myeloma, malignant giant cell tumor chordoma, osteochronfroma, benign chondroma, chondroblastoma, chondromyxofibroma, osteoid osteoma, giant cell tumors, cancer of the skuli, meninges cancer, brain cancer, spinal cord cancer, uterus cancer, cervical cancer, cancer of the ovaries, vulva cancer, vagina cancer, Hodgkin's disease, non-Hodgkin's lymphoma, malignant melanoma, basal cell carcinoma, squamous cell carcinoma, Karposi's sarcoma, moles dysplastic nevi, lipoma, angioma, and dermatofibroma.

In some embodiments, the chemotherapeutic agent is selected from the group consisting of doxorubicin, cisplatin, oxaliplatin, rituximab, hydroxyurea or trastuzumab.

In one aspect of the invention, there is a method for determining suitability of a compound to (i) treating or preventing development of cancer and/or (ii) increasing or prolonging the therapeutic efficacy of a chemotherapeutic agent, the method comprising contacting the compound with a pocket domain within a region of a Mitotic Arrest Deficient 2 Like 2 protein (MAD2L2; SEQ ID NO: 1), wherein binding of the compound to the pocket is indicative of the compound being effective in (i) treating or preventing development of cancer and/or (ii) increasing or prolonging the therapeutic efficacy of a chemotherapeutic agent.

In some embodiments, the binding is to one or more of: SEQ ID NO: 2 (LLAAFILK); and SEQ ID NO: 3 (LIPLKTMTSDILKMQLYV).

In some embodiments, the binding is determined by inhibition of MAD2L2:Rev1 interaction.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 Presents the chemical structure of compounds 2, 3, 4 and 9 used in the cell survival assays;

FIG. 2 presents a graph of colony survival assay on U2OS cells treated with varying concentrations of cisplatin without added inhibitor (DMSO), with compound 2 as inhibitor (Compound #2), with compound 3 as inhibitor (Compound #3) and with compound 9 as inhibitor (Compound #9);

FIG. 3 presents a graph of colony survival assay on A549 cells treated with varying concentrations of cisplatin without added inhibitor (DMSO), with compound 2 as inhibitor (Compound #2) and with compound 3 as inhibitor (Compound #3);

FIG. 4 presents a graph of colony survival assay on H1975 cells treated with varying concentrations of cisplatin without added inhibitor (DMSO), with compound 2 as inhibitor (Compound #2) and with compound 3 as inhibitor (Compound #3);

FIG. 5 presents a picture of the anti-GFP immunoprecipitation (Co-IP) assay in HEK293 cells with 50 μM applied compounds 2, 3, and DMSO as negative control;

FIG. 6 presents a picture of the anti-GFP immunoprecipitation (Co-IP) assay in HEK293 cells with 50 μM applied compounds 2, 3, 4 and DMSO as negative control;

FIG. 7 presents a graphical summary of results from two Co-IP experiments in HEK293 cells, with 50 μM applied compounds 2, 3 and 4. Error bars show standard deviation. For DMSO, 2, and 3, n=2. For 4, n=1;

FIGS. 8A-8B present MAD2L2 dimer in complex with a CAMP fragment and Rev1: Proteins are shown in cartoon representation: MAD2L2 (yellow and green), CAMP fragment (orange and cyan) and Rev1 (wheat) (FIG. 8A). Surface representation of the cavity between the MAD2L2 monomers after binding Rev1. Rev1 monomers are colored yellow and green and K190 is shown in magenta. Surface representation of the cavity between MAD2L2 monomers colored as above. Residue K190 of each monomer is colored magenta (FIG. 8B);

FIG. 9 presents small molecules docked in the cavity of the MAD2L2/REV7 dimer: the small molecules ZINC97017995 and ZINC25496030, docked in the cavity of the MAD2L2 dimer, are shown using stick representation and colored purple and hot pink, respectively. The protein complex is shown using cartoon representation. Rev1 was removed from the complex before docking; however, it is shown in a semi-transparent cartoon for clarification.

FIG. 10 is a bar graph showing compounds #2 and #3 present no toxicity. H1975 and A549 cell lines presented no reduction in cell viability after performing the colony survival assay with 50 μM of each compound alone;

FIGS. 11A-11C are graphs showing compounds #2 and #3 sensitize cells to cisplatin treatment: colony survival assay of A549 cell line (FIG. 11A); colony survival assay of H1975 cell line (FIG. 11B). Both cell lines were treated with 50 μM of compound and the indicated concentration of cisplatin for 48 h, then growth media was replaced with normal growth media with no compound. Cells were allowed to recover 4-5 days before staining. For both cell lines, colony survival assay n=3 independent experiments, SD=1. P value was calculated by two-way ANOVA multiple comparison. Colony survival assay of H1975 cell line (FIG. 11C). Cells were treated with 50 μM of compound and the indicated concentration of doxorubicin for 48 h, then growth media was replaced with normal growth media with no compound. Cells were allowed to recover 4-5 days before staining. n=3 independent experiments, SD=1. P value was calculated by two tailed t-test;

FIG. 12A-12C present compounds #2 and #3 increase DNA damage after combined treatment: representative figures of γH2AX in A549 (left panel) cells and H1975 (right panel) with different treatments as indicated (FIG. 12A); quantification of A549 and H1975 cells foci/cell in each treatment (FIG. 12B). The total number of cells scored is indicated. Error bars=1 SD. P-value was calculated by two-tailed t-test. Quantification of A549 (left panel) and H1975 (right panel) cells with more than 80 foci/cell in each treatment (FIG. 12C). The total number of cells scored is indicated. These data were derived from four captures, in each of which at least 400 cells were scored. SD=1, for the independently determined percentages from the two experiments. For A549 cells p-value was calculated by two-tailed t-test and for H1975 cells p-value was calculated by one-tailed t-test. A549 cells were treated with 10 μM cisplatin and H1975 cells with 5 μM cisplatin. Both cell lines were treated with 50 μM of c #2 or c #3; and

FIGS. 13A-13G present compounds #2 and #3 reduce Rev1-MAD2L2 interaction: binding activity of the compounds c #2 and c #3 to MAD2L2 in vitro (FIG. 13A). Binding assay of increasing concentrations of compounds c #2 and c #3 to MAD2L2. c #2 KD=310.8±32.96 μM; c #3 KD=44.88±2.062 μM. MAD2L2's cellular thermal shift assay (CETSA) in 293T cells after 1-hour treatment with DMSO or 50 μM of c #2 or c #3 (FIG. 13B). Quantification of the relative amount of MAD2L2 in each cellular thermal shift assay (FIG. 13C). n=three independent experiments. P-value was calculated by two-way ANOVA multiple comparisons. The additional blots contributing to this analysis are shown in FIG. 13F. HEK293 cells were co-transfected with YFP-Rev1 together with myc-MAD2L2, and an anti-GFP IP was performed to assess Rev1-MAD2L2 binding when treated with 50 μM of each compound (FIG. 13D). Quantification of the relative amount of myc-MAD2L2 bound to YFP-Rev1 (FIG. 13E). For DMSO and c #3 n=three independent experiments, for c #2 n=two independent experiments, SD=1. P-value was calculated by two-tailed t-test. The additional co-IP blots contributing to this analysis are shown in FIG. 13G.

FIGS. 14A-14D present graphs showing cancer cells sensitization to cisplatin treatment bz compound 3 (c #3). (14A) C#3 caused no toxicity during the colony survival assay. (14B) Colony survival assay of H1975 cell line. (14C) Colony survival assay of A549 cell line. (14D) Colony survival assay of B16F10 cell line. All cell lines were treated with 50 μM c #3 and the indicated concentration of cisplatin for 48 h, then growth media was replaced with normal growth media with no compound. Cells were allowed to recover 4-5 days before staining. For all cell lines, colony survival assay n=3 independent experiments, SD=1. P value was calculated by two-way ANOVA multiple comparison. IC50 was calculated using linear regression for H1975 and A549, and sigmoidal regression for B16F10.

FIGS. 15A-15B3 present micrographs and graphs showing that c #3 increases DNA damage after combined treatment. (15A). Representative figures of TH2AX in A549 cells (left panel), H1975 (middle panel) and B16F10 (right panel) with different treatments as indicated. Quantification of A549 (15B1), H1975(15B2), and B16F10 cells(15B3)′ foci/cell in each treatment. The total number of cells scored is indicated. Error bars=1 SD, P-value was calculated by two-tailed t-test. In all experiments, A549 cells were treated with 10 μM cisplatin and H1975 and B16F10 cells with 5 μM cisplatin. All cell lines were co-treated with 50 μM of c #3. Cells were co-treated for 16 h before analysis.SD=1, The P-value was calculated using a one-tailed t-test.

FIG. 16 is a bar graph showing that c #3 reduces Rev1-MAD2L2 interaction. PLA on cells treated with 10 μM cisplatin and 100 μM of c #3 for 24 h. In each analyzed field, cells and foci were counted separately and the ratio of foci/cell was calculated. Each dot represents the average of foci/cell in one analyzed field. SD=1. The P-value was calculated by a one-tailed t-test.

FIGS. 17A-17E are micrographs and graphs showing that C#3 prevents tumor growth. 2*106 B16F10 cells were injected to the left flank of C₅₇BL mice. After three days different treatments, as indicated, were injected to the newly formed tumor. (17A) tumor size (mm3) was measured daily for each mouse. In the co-treatment, almost no change in tumor size was observed, in the first three days. (17B) representative images of three tumors from each treatment group. (17C) For each treatment the fold-change in the volume of each day was compared to day 0. No change was observed in the co-treated group during the first three days. (17D) The co-treated group presented less developed tumors, which comprised about 2-3% of mice weight. (17E) H&E staining of a representative tumor from each group. Number of mice for each group in this experiment: DMSO n=7, cisplatin n=10, cisplatin+c #3 n=10, c #3 n=9. SD=1. P-values were calculated by one-tailed t-test.

### DETAILED DESCRIPTION OF THE INVENTION

According to some embodiments, the present invention provides a pharmaceutical composition comprising any of the compounds as described herein. In some embodiments the compound targets protein-protein interactions. In some embodiments, the compound inhibits translesion DNA synthesis (TLS).

In some embodiments, the pharmaceutical composition is for use in combination therapy with a chemotherapeutic agent.

According to some embodiments, a pharmaceutically composition comprising a compound as described herein, comprises a pharmaceutically acceptable carrier, adjuvant or vehicle. Non-limiting examples of the pharmaceutically acceptable carrier, adjuvant, or vehicle, according to the present invention include solvents, diluents, or other liquid vehicle, dispersion or suspension aids, surface active agents, isotonic agents, thickening or emulsifying agents, preservatives, solid binders, lubricants and the like.

According to some embodiments, the present invention provides a method for increasing or prolonging the therapeutic efficacy of a chemotherapeutic agent in a subject in need thereof.

According to some embodiments, the present invention provides a method for treating or preventing development of cancer in a subject in need thereof.

The present invention is also directed to an agent that binds a region of a Mitotic Arrest Deficient 2 Like 2 protein (MAD2L2).

### The Composition

According to some embodiments, the present invention provides a pharmaceutical composition comprising a compound, pharmaceutically acceptable salt, isomer or tautomer thereof, wherein the compound is represented by Formula I:

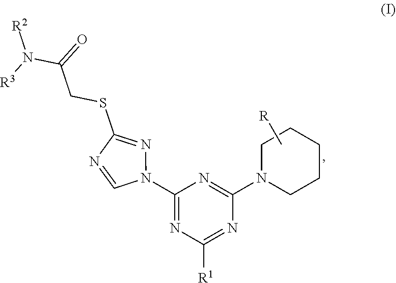

wherein: R, R¹, R² and R³ are independently selected from the group consisting of an optionally substituted heteroaryl, an optionally substituted aryl, a optionally substituted bicyclic aromatic ring, an optionally substituted aliphatic ring, an optionally substituted unsaturated aliphatic ring, an optionally substituted bicyclic aliphatic ring, an optionally substituted linear C₁-C₇ alkyl group, an optionally substituted branched C₁-C₇ alkyl group, an optionally substituted branched C₁-C₇ haloalkyl group, an optionally substituted linear C₁-C₇ haloalkyl group, an optionally substituted C₁-C₇ alkylhydroxy group, an optionally substituted C₁-C₇ alkoxy group, a hydrogen, a heteroatom, a hydroxy group, a halo group, an alkyl group, an alkoxy group, an amino group, or any combination thereof.

In some embodiments, R¹ represents a substituent comprising any one of —NO₂, —CN, —OH, —NH₂, carbonyl, —CONH₂, —CONR₂, —CNNR₂, —CSNR₂, —CONH—OH, —CONH—NH₂, —NHCOR, —NHCSR, —NHCNR, —NC(═O)R, —NC(═O)OR, —NC(═O)NR, —NC(═S)OR, —NC(═S)NR, —SO₂R, —SOR, —SR, —SO₂OR, —SO₂N(R)₂, —NHNR₂, —NNR, C₁-C₇ haloalkyl, optionally substituted C₁-C₁₀ alkyl, —NH(C₁-C₆ alkyl), —N(C₁-C₆ alkyl)₂, C₁-C₆ alkoxy, C₁-C₆haloalkoxy, hydroxy(C₁-C₆ alkyl), hydroxy(C₁-C₆ alkoxy), alkoxy(C₁-C₆ alkyl), alkoxy(C₁-C₆ alkoxy), amino(C₁-C₆ alkyl), —CONH(C₁-C₆ alkyl), —CON(C₁-C₆ alkyl)₂, —CO₂H, —CO₂R, —OCOR, —C(═O)R, —OC(═O)OR, —OC(═O)NR, —OC(═S)OR, —OC(═S)NR, a heteroatom, an optionally substituted cycloalkyl, an optionally substituted heterocyclyl, an optionally substituted aryl, or a combination thereof.

In some embodiments, the compound is:

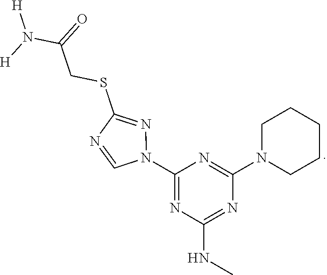 [SMILES: CNc1nc(N2CCCCC2)nc(-n2cnc(SCC(N)=O)n2)n1]

In some embodiments, the compound is:

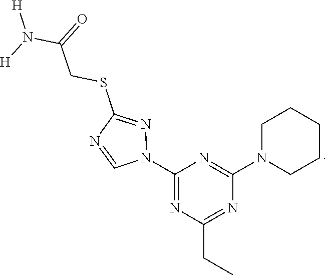 [SMILES: CCc1nc(N2CCCCC2)nc(-n2cnc(SCC(N)=O)n2)n1]

According to some embodiments, the present invention provides a pharmaceutical composition comprising a compound, pharmaceutically acceptable salt, isomer or tautomer thereof, wherein the compound is represented by Formula II:

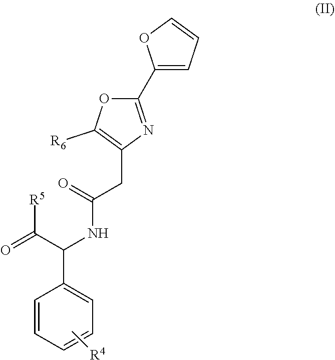

wherein: R⁴, R⁵ and R⁶ are independently selected from the group consisting of a heteroaryl, an aryl, a bicyclic aromatic ring, an aliphatic ring, an unsaturated aliphatic ring, a bicyclic aliphatic ring, a linear C₁-C₇ alkyl group, a branched C₁-C₇ alkyl group, a branched C₁-C₇ haloalkyl group, a linear C₁-C₇ haloalkyl group, a C₁-C₇ alkylhydroxy group, a C₁-C₇ alkoxy group, a hydrogen, a hydroxy group, a halo group, an alkyl group, an alkoxy group, an amino group, or any combination thereof.

In some embodiments, the compound is:

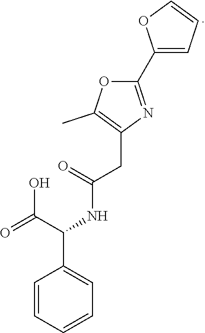 [SMILES: Cc1oc(-c2ccco2)nc1CC(=O)N[C@@H](C(=O)O)c1ccccc1]

According to some embodiments, the present invention provides a pharmaceutical composition comprising a compound, pharmaceutically acceptable salt, isomer or tautomer thereof, wherein the compound is represented by Formula III:

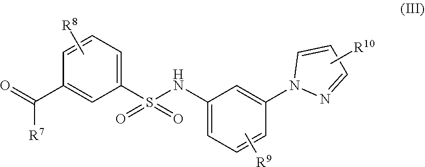

wherein: R⁷, R⁸, R⁹ and R¹⁰ are independently selected from the group consisting of a heteroaryl, an aryl, a bicyclic aromatic ring, an aliphatic ring, an unsaturated aliphatic ring, a bicyclic aliphatic ring, a linear C₁-C₇ alkyl group, a branched C₁-C₇ alkyl group, a branched C₁-C₇ haloalkyl group, a linear C₁-C₇ haloalkyl group, a C₁-C₇ alkylhydroxy group, a C₁-C₇ alkoxy group, a hydrogen, a hydroxy group, a halo group, an alkyl group, an alkoxy group, an amino group, or any combination thereof.

In some embodiments, the compound is:

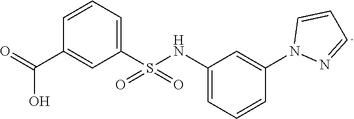 [SMILES: O=C(O)c1cccc(S(=O)(=O)Nc2cccc(-n3cccn3)c2)c1]

According to some embodiments, the present invention provides a pharmaceutical composition comprising a compound, pharmaceutically acceptable salt, isomer or tautomer thereof, wherein the compound is represented by Formula IV:

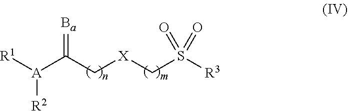

wherein: A is selected from O, N, and CH; Ba and Bb are independently selected from the group consisting of NH, NH₂, S, SH, O, OH, CH₂ and CH; n and m independently represent an integer in a range from 0 to 10; X represents a heteroatom or aryl; R¹, R², are absent or independently selected from the group consisting of hydrogen, C₁-C₇ alkyl group, or a branched C₁-C₇ alkyl group, an aryl, a bicyclic aromatic ring; and R³ represents a heteroaryl, an aryl, a bicyclic aromatic ring, an aliphatic ring, an unsaturated aliphatic ring, a bicyclic aliphatic ring, linear C₁-C₇ alkyl group, a branched C₁-C₇ alkyl group a branched C₁-C₇ haloalkyl group, a linear C₁-C₇ haloalkyl group, a C₁-C₇ alkylhydroxy group, a C₁-C₇ alkoxy group, hydrogen, hydroxy group, a halo group, an alkyl group, an alkoxy group, an amino group, a guanidine group, a thioalkoxy group, a a mercapto group, a cyano group, a haloalkyl group, a nitro group, an azo group, a sulfonate group, a sulfinyl group, a vinyl group, an allyl group, a thioalkyl group, an alkylhydroxy group, a keto group, an ether, and a sulfone group or any combination thereof.

In some embodiments, the compound is represented by Formula V or Formula VI:

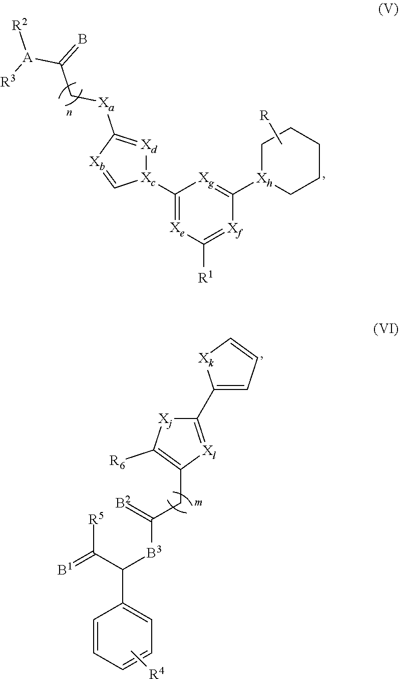

wherein: A is selected from N, and CH; B, B¹, B² and B³ are independently selected from the group consisting of NH, NH₂, S, SH, O, OH, CH₂ and CH; Xₐ-Xₖ independently represent a heteroatom; n and m independently represent an integer in a range from 0 to 10; and R, R¹, R² and R³ are independently selected from the group consisting of an optionally substituted heteroaryl, an optionally substituted aryl, a optionally substituted bicyclic aromatic ring, an optionally substituted aliphatic ring, an optionally substituted unsaturated aliphatic ring, an optionally substituted bicyclic aliphatic ring, an optionally substituted linear C₁-C₇ alkyl group, an optionally substituted branched C₁-C₇ alkyl group, an optionally substituted branched C₁-C₇ haloalkyl group, an optionally substituted linear C₁-C₇ haloalkyl group, an optionally substituted C₁-C₇ alkylhydroxy group, an optionally substituted C₁-C₇ alkoxy group, a hydrogen, a heteroatom, a hydroxy group, a halo group, an alkyl group, an alkoxy group, an amino group, or any combination thereof.

According to some embodiments, the present invention provides a pharmaceutical composition comprising a compound, pharmaceutically acceptable salt, isomer or tautomer thereof, wherein the compound is represented by Formula VII:

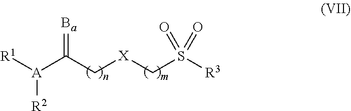

wherein: A is selected from O, N, and CH; Ba is selected from the group consisting of NH, NH₂, S, SH, O, OH, CH₂ and CH; n and m independently represent an integer in a range from 0 to 10; X represents a heteroatom or aryl; R¹, R², are absent or independently selected from the group consisting of hydrogen, C₁-C₇ alkyl group, or a branched C₁-C₇ alkyl group, an aryl, a bicyclic aromatic ring; and R³ represents a heteroaryl, an aryl, a bicyclic aromatic ring, an aliphatic ring, an unsaturated aliphatic ring, a bicyclic aliphatic ring, linear C₁-C₇ alkyl group, a branched C₁-C₇ alkyl group a branched C₁-C₇ haloalkyl group, a linear C₁-C₇ haloalkyl group, a C₁-C₇ alkylhydroxy group, a C₁-C₇ alkoxy group, hydrogen, hydroxy group, a halo group, an alkyl group, an alkoxy group, an amino group, a guanidine group, a thioalkoxy group, a a mercapto group, a cyano group, a haloalkyl group, a nitro group, an azo group, a sulfonate group, a sulfinyl group, a vinyl group, an allyl group, a thioalkyl group, an alkylhydroxy group, a keto group, an ether, and a sulfone group or any combination thereof.

In some embodiments, the compound is represented by Formula VIII:

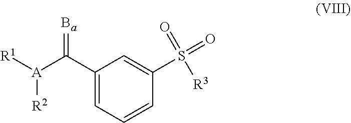

wherein: A is selected from O, N, and CH; Ba is selected from the group consisting of NH, NH₂, S, SH, O, OH, CH₂ and CH; R¹, R², are absent or independently selected from the group consisting of hydrogen, C₁-C₇ alkyl group, or a branched C₁-C₇ alkyl group, an aryl, a bicyclic aromatic ring; and R³ represents a heteroaryl, an aryl, a bicyclic aromatic ring, an aliphatic ring, an unsaturated aliphatic ring, a bicyclic aliphatic ring, linear C₁-C₇ alkyl group, a branched C₁-C₇ alkyl group a branched C₁-C₇ haloalkyl group, a linear C₁-C₇ haloalkyl group, a C₁-C₇ alkylhydroxy group, a C₁-C₇ alkoxy group, hydrogen, hydroxy group, a halo group, an alkyl group, an alkoxy group, an amino group, a guanidine group, a thioalkoxy group, a a mercapto group, a cyano group, a haloalkyl group, a nitro group, an azo group, a sulfonate group, a sulfinyl group, a vinyl group, an allyl group, a thioalkyl group, an alkylhydroxy group, a keto group, an ether, and a sulfone group or any combination thereof.

In some embodiments, a compound is represented by Formula I, II, III, IV, V, VI, VII, or VIII, wherein R¹, R², R³, R⁴, R⁵, R⁶, R⁷, R⁸, R⁹ and R¹⁰ R, R¹, R² and R³ are independently selected from the group consisting of an optionally substituted heteroaryl, an optionally substituted aryl, a optionally substituted bicyclic aromatic ring, an optionally substituted aliphatic ring, an optionally substituted unsaturated aliphatic ring, an optionally substituted bicyclic aliphatic ring, an optionally substituted linear C₁-C₇ alkyl group, an optionally substituted branched C₁-C₇ alkyl group, an optionally substituted branched C₁-C₇ haloalkyl group, an optionally substituted linear C₁-C₇ haloalkyl group, an optionally substituted C₁-C₇ alkylhydroxy group, an optionally substituted C₁-C₇ alkoxy group, a hydrogen, a heteroatom, a hydroxy group, a halo group, an alkyl group, an alkoxy group, an amino group, or any combination thereof.

In some embodiments, a compound is represented by Formula I, II, III, IV, V, VI, VII, or VIII, wherein R¹, R², R³, R⁴, R⁵, R⁶, R⁷, R⁸, R⁹ and R¹⁰ R, R¹, R² and R³ are absent or independently selected from the group consisting of a heteroaryl, an aryl, a bicyclic aromatic ring, an aliphatic ring, an unsaturated aliphatic ring, a bicyclic aliphatic ring, linear C₁-C₇ alkyl group, a branched C₁-C₇ alkyl group a branched C₁-C₇ haloalkyl group, a linear C₁-C₇ haloalkyl group, a C₁-C₇ alkylhydroxy group, a C₁-C₇ alkoxy group, hydrogen, hydroxy group, a halo group, an alkyl group, an alkoxy group, an amino group, a guanidine group, a thioalkoxy group, a a mercapto group, a cyano group, a haloalkyl group, a nitro group, an azo group, a sulfonate group, a sulfinyl group, a vinyl group, an allyl group, a thioalkyl group, an alkylhydroxy group, a keto group, an ether, and a sulfone group, hydroxy(C₁-C₆ alkyl), hydroxy(C₁-C₆ alkoxy), alkoxy(C₁-C₆ alkyl), alkoxy(C₁-C₆ alkoxy), amino(C₁-C₆ alkyl), CONH₂, CONH(C₁-C₆ alkyl), CON(C₁-C₆ alkyl)₂, CONH—OH, CO₂H, CO₂(C₁-C₆ alkyl), SO₂R, SO₂OR, SO₂N(R)₂, cyclopropylethynyl, substituted aryl, substituted heteroaryl, substituted heterocyclyl, substituted C₃-C₅ cycloalkyl, substituted heterocyclyl, substituted aryloxy, substituted heteroaryloxy, and any combination thereof.

In some embodiments, the compound of the invention is as represented by an of the Formulae disclosed herein, and wherein the compound is characterized by sufficient biological activity, as disclosed herein such as being characterized by binding affinity to MAD2L2 sufficient for preventing MAD2L2-Rev1 complex formation.

In some embodiments, the pharmaceutical composition is for use in combination therapy with a chemotherapeutic agent. In some embodiments, the chemotherapeutic agent is selected from the group consisting of a crosslinking agent, a strand break agent, an alkylating agent, an anti-metabolite agent, a microtubule disruptor, a radiomimetic agent, a radiosensitizer, an intercalator, a DNA replication inhibitor, an anthracycline, an etoposide, and a topoisomerase II inhibitor.

In some embodiments, the chemotherapeutic agent is a DNA damaging agent (and/or an agent capable of stably binding to DNA).

Non-limiting examples of DNA damaging agents according to the present invention include agents such as topoisomerase inhibitors (e.g., etoposide, ramptothecin, topotecan, teniposide, mitoxantrone), anti-microtubule agents (e.g., vincristine, vinblastine), anti-metabolic agents (e.g., cytarabine, methotrexate, hydroxyurea, 5-fluorouracil, floxuridine, 6-thioguanine, 6-mercaptopurine, fludarabine, pentostatin, chlorodeoxyadenosine), DNA alkylating agents (e.g., cisplatin, mechlorethamine, cyclophosphamide, ifosfamide, melphalan, chorambucil, busulfan, thiotepa, carmustine, lomustine, carboplatin, dacarbazine, procarbazine), DNA strand break inducing agents (e.g., bleomycin, doxorubicin, daunorubicin, idarubicin, mitomycin C), and radiation therapy.

In some embodiments, the chemotherapeutic agent is selected from the group consisting of cisplatin, carboplatin, etoposide, oxaliplatin, rituximab or trastuzumab, mechlorethamine, cyclophosphamide, bleomycin, doxorubicin, daunorubicin, cytarabine, methotrexate, hydroxyurea, or a combination thereof.

In some embodiments, the compound and the chemotherapeutic agent are present in the composition at a weight to weight (w/w) ratio ranging from 1:0.01 to 0.01:1, 1:0.09 to 0.01:1, 1:0.1 to 0.01:1, 1:0.5 to 0.01:1, 1:0.9 to 0.01:1, 1:1 to 0.01:1, 1:0.09 to 0.02:1, 1:0.1 to 0.02:1, 1:0.5 to 0.02:1, 1:0.9 to 0.02:1, 1:1 to 0.02:1, 1:0.09 to 0.05:1, 1:0.1 to 0.05:1, 1:0.5 to 0.05:1, 1:0.9 to 0.05:1, 1:1 to 0.05:1, 1:0.09 to 0.09:1, 1:0.1 to 0.09:1, 1:0.5 to 0.09:1, 1:0.9 to 0.09:1, 1:1 to 0.09:1, 1:0.09 to 0.1:1, 1:0.1 to 0.1:1, 1:0.5 to 0.1:1, 1:0.9 to 0.1:1, 1:1 to 0.1:1, 1:0.09 to 0.5:1, 1:0.1 to 0.5:1, 1:0.5 to 0.5:1, 1:0.9 to 0.5:1, or 1:1 to 0.5:1, including any range therebetween.

In some embodiments, the pharmaceutical composition further comprises a pharmaceutically acceptable carrier.

In some embodiments, the pharmaceutical composition is for use in the prevention or treatment of a disorder associated with cancer.

In some embodiments, the pharmaceutical composition is for use in increasing the sensitivity of cancer cells to a cancer therapy selected from chemotherapy or radiation therapy.

In some embodiments, the pharmaceutical composition is for use in sensitizing cancer cells, promoting cell death in cancer cells, or inhibiting cell repair from DNA damage.

In some embodiments, the pharmaceutical composition targets protein-protein interactions. In some embodiments a compound as described herein targets protein-protein interactions. In some embodiments, a compound as described herein targets Rev1-Rev7 (MAD2L2) binding interaction.

As used herein “Rev1” and “Rev7” refer to pivotal proteins in translesion DNA synthesis, which allows DNA synthesis even in the presence of DNA damage. In some embodiments, the Rev1 C-terminal domain adopts a four-helix bundle that interacts with REV7.

In some embodiments, the pharmaceutical composition is for use in a patient. In some embodiments, the pharmaceutical composition is for use in cancer cells, wherein the cells are derived from H-1975 cells, U2OS cells, A549 cells, HEK293 cells, or any combination thereof.

### Mitotic Arrest Deficient 2 Like 2 Protein (MAD2L2) Targeting Agents

According to some embodiments, the present invention provides an agent that binds a region of a Mitotic Arrest Deficient 2 Like 2 protein

(MAD2L2; SEQ ID NO: 1; MGSSHHHHHHSQDPNSMTTLTRQDLNF
GQVVADVLCEFLEVAVHLILYVREVYPVGIFQKRKKYNVPVQMSCHPELN
QYIQDTLHCVKPLLEKNDVEKVVVVILDKEHRPVEKFVFEITQPPLLSIS
SDSLLSHVEQLLAAFILKISVCDAVLDHNPPGCTFTVLVHTREAATRNME
KIQVIKDFPWILADEQDVHMHDPRLIPLKTMTSDILKMQLYVEERAHKG
S).

As used herein, MAD2L2 relates to Mitotic Arrest Deficient 2 Like 2 protein having UniProt Accession no. Q9UI95.

In some embodiments, the agent binds at least one region of MAD2L2 selected from any one of: SEQ ID NO: 2 (LLAAFILK); and SEQ ID NO: 3 (LIPLKTMTSDILKMQLYV), or a homolog thereof.

In some embodiments, the agent binds an amino acid residue selected from residues L122, A125, F126, K129, L186, 1187, P188, L189, K190, T191, S194 of MAD2L2 (SEQ ID NO:1). In some embodiments, the agent binds a combination (i.e. a plurality) of amino acid residues selected from residues L122, A125, F126, K129, L186, 1187, P188, L189, K190, T191, S194 of MAD2L2 (SEQ ID NO:1).

In some embodiments, the agent binds at least one amino acid residue selected from residues L128, T191, T193, S194, L197, K198, M199, Q200, L201 of MAD2L2 (SEQ ID NO:1). In some embodiments, the agent binds a combination (i.e. a plurality) of amino acid residues selected from residues L128, T191, T193, S194, L197, K198, M199, Q200, L201 of MAD2L2 (SEQ ID NO:1).

In some embodiments, the agent binds one or more amino acid residue selected from L122, A125, F126, K129, L186, 1187, P188, L189, K190, T191, S194 of MAD2L2 (SEQ ID NO:1) and L128, T191, T193, 5194, L197, K198, M199, Q200, L201 of MAD2L2 (SEQ ID NO:1). In some embodiments, the agent binds a plurality (e.g. 2, 3, 4, 5, 6, 8, 10, between 2 and 21, between 5 and 10, between 5 and 21, including any range between) of amino acid residue selected from L122, A125, F126, K129, L186, 1187, P188, L189, K190, T191, S194 of MAD2L2 (SEQ ID NO:1) and L128, T191, T193, 5194, L197, K198, M199, Q200, L201 of MAD2L2 (SEQ ID NO:1).

As used herein, a homolog of SEQ ID NO: 2 (LLAAFILK); and SEQ ID NO: 3 (LIPLKTMTSDILKMQLYV), refers to at least one mutation (e.g., substitution) for which the agent can still bind a pocket region a Mitotic Arrest Deficient 2 Like 2 protein (MAD2L2; SEQ ID NO: 1), and provide the desired biological or pharmaceutical effect (e.g., hinder or inhibit a MAD2L2:Rev1 interaction or inhibits inter-MAD2L2 interactions).

Non-limiting examples for identifying the pocket include the following algorithms utilized by GROMACS, Modeller, SiteMap, FtSite, or fPocket. In some embodiments, the pocket is identified using GROMACS, or Modeller program.

As used herein, the term “pocket” refers to a cavity, indentation or depression in the surface of a protein molecule that is created as a result of the folding of the peptide chain into the 3-dimensional structure that makes the protein functional. A pocket can readily be recognized by inspection of the protein structure and/or by using commercially available modeling software's.

The term “agent” as used herein refers to any small organic molecule capable of entering and/or binding to a protein pocket as described hereinabove.

As used herein, the term “small organic molecule” refers to a molecule of a size comparable to those organic molecules generally used in pharmaceuticals. The term excludes natural biological macromolecules (e.g., proteins, nucleic acids, etc.). In some embodiments, organic molecules have a size up to 5000 Da, up to 2000 Da, or up to 1000 Da, including any value therebetween. Each possibility represents a separate embodiment of the invention.

In some embodiments, the agent is a compound represented by Formula I, Formula II, or Formula III.

In some embodiments, the agent is selected from the group consisting of:

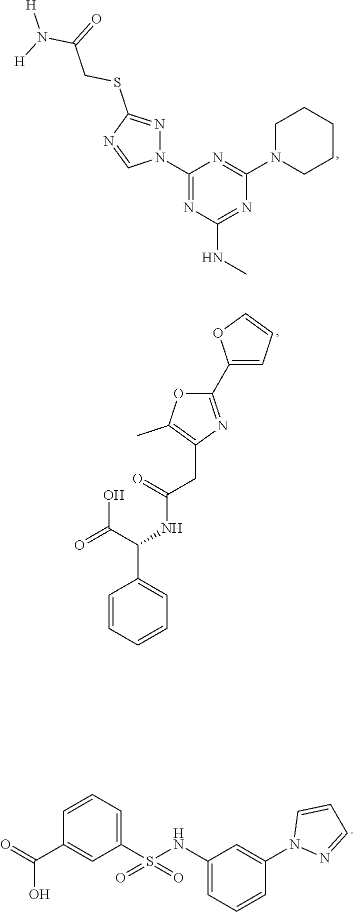

In some embodiments, the binding is specific binding.

The terms “specific binding” or “preferential binding” refer to that binding which occurs between two paired species (such as enzyme/substrate, receptor/agonist, antibody/antigen, and lectin/carbohydrate) which may be mediated by covalent and/or non-covalent interactions. When the interaction of the two species typically produces a non-covalently bound complex, the binding which occurs is typically electrostatic, and/or hydrogen-bonding, and/or the result of lipophilic interactions. Accordingly, “specific binding” occurs between pairs of species where there is interaction between the two that produces a bound complex. In particular, the specific binding is characterized by the preferential binding of one member of a pair to a particular species as compared to the binding of that member of the pair to other species within the family of compounds to which that species belongs. Thus, for example, an agent may show an affinity for a particular pocket on a MAD2L2 molecule (i.e., the pocket defined herein) that is at least two-fold preferably, at least 10 fold, at least 100 fold, at least 1000 fold, or at least 10000 fold greater than its affinity for a different pocket on the same or related proteins, including any value therebetween. Each possibility represents a separate embodiment of the invention.

In some embodiments, the agent inhibits a MAD2L2:Rev1 interaction. In some embodiments, the agent inhibits inter-MAD2L2 interactions.

In some embodiments, the agent is for use in the prevention or treatment of a disorder associated with cancer.

In some embodiments, the agent is for use in increasing the sensitivity of cancer cells to a cancer therapy selected from chemotherapy or radiation therapy.

In some embodiments, the agent is for use in sensitizing cancer cells, promoting cell death in cancer cells, or inhibiting cell repair from DNA damage.

The disclosed agents, alone or in combination thereof or with any another therapeutically active agent, can be designed and utilized to exert a dual and possibly synergistic activity when in combination thereof or with any another therapeutically active agent.

According to some embodiments, the present invention provides a pharmaceutical composition comprising the agent described hereinabove.

In some embodiments, the pharmaceutical composition comprises between 100 nM and 5 mM, between 150 nM and 5 mM, between 200 nM and 5 mM, between 500 nM and 5 mM, between 700 nM and 5 mM, between 900 nM and 5 mM, between 1 mM and 5 mM, between 2 mM and 5 mM, between 100 nM and 3 mM, between 150 nM and 3 mM, between 200 nM and 3 mM, between 500 nM and 3 mM, between 700 nM and 3 mM, between 900 nM and 3 mM, between 1 mM and 3 mM, between 2 mM and 3 mM, between 100 nM and 1 mM, between 150 nM and 1 mM, between 200 nM and 1 mM, between 500 nM and 1 mM, or between 700 nM and 1 mM, of the agent, including any range therebetween. Each possibility represents a separate embodiment of the invention.

According to some embodiments, the present invention provides a method for increasing or prolonging the therapeutic efficacy of a chemotherapeutic agent in a subject in need thereof, comprising administering to the subject a therapeutically effective amount of the pharmaceutical composition described hereinabove.

According to some embodiments, the present invention provides a method for treating or preventing development of cancer in a subject in need thereof, comprising administering to the subject a therapeutically effective amount of (a) chemotherapeutic agent and (b) the pharmaceutical composition described hereinabove.

In some embodiments, the cancer is selected from the group consisting of lung cancer, breast cancer, osteosarcoma, neuroblastoma, colon adenocarcinoma, chronic myelogenous leukemia (CML), acute myeloid leukemia (AML), acute promyelocyte leukemia (APL), sarcoma, myxoma, rhabdomyoma, fibroma, lipoma, teratoma; bronchogenic carcinoma, alveolar carcinoma, bronchial adenoma, sarcoma, lymphoma, chondromatous hamartoma, mesothelioma, esophageal cancer, stomach cancer, pancreatic cancer, small bowel cancer, large bowel cancer; kidney cancer, bladder cancer, urethra cancer, prostate cancer, testis cancer; hepatoma, cholangiocarcinoma, hepatoblastoma, angiosarcoma, hepatocellular adenoma, hemangioma, osteogenic sarcoma, fibrosarcoma, malignant fibrous histiocytoma, chondrosarcoma, Ewing's sarcoma, malignant lymphoma, multiple myeloma, malignant giant cell tumor chordoma, osteochronfroma, benign chondroma, chondroblastoma, chondromyxofibroma, osteoid osteoma, giant cell tumors, cancer of the skuli, meninges cancer, brain cancer, spinal cord cancer, uterus cancer, cervical cancer, cancer of the ovaries, vulva cancer, vagina cancer, Hodgkin's disease, non-Hodgkin's lymphoma, malignant melanoma, basal cell carcinoma, squamous cell carcinoma, Karposi's sarcoma, moles dysplastic nevi, lipoma, angioma, and dermatofibroma.

In some embodiments, the chemotherapeutic agent is selected from the group consisting of doxorubicin, cisplatin, oxaliplatin, rituximab, hydroxyurea or trastuzumab.

### Screening Method

According to an aspect of some embodiments of the present invention, there is provided a method for determining suitability of a compound to (i) treating or preventing development of cancer and/or (ii) increasing or prolonging the therapeutic efficacy of a chemotherapeutic agent, the method comprising contacting the compound with a pocket domain within a region of a Mitotic Arrest Deficient 2 Like 2 protein (MAD2L2; SEQ ID NO: 1), wherein binding of the compound to the pocket is indicative of the compound being effective in (i) treating or preventing development of cancer and/or (ii) increasing or prolonging the therapeutic efficacy of a chemotherapeutic agent.

In some embodiments, the binding is to one or more of: SEQ ID NO: 2 (LLAAFILK); and SEQ ID NO: 3 (LIPLKTMTSDILKMQLYV).

In some embodiments, the binding is determined by inhibition of MAD2L2:Rev1 interaction.

In some embodiments, the method comprises a step of computational screening of libraries of compounds.

Further embodiments of the disclosed method are provided in the Examples section below.

### The Method

According to some embodiments, the present invention provides a method for increasing or prolonging the therapeutic efficacy of a chemotherapeutic agent in a subject in need thereof, comprising administering to the subject a therapeutically effective amount of a pharmaceutical composition described herein above.

According to one aspect, there is provided a method for treating a subject afflicted with cancer, the method comprising the steps of administering to the subject a therapeutically effective amount of: (a) chemotherapeutic agent and (b) a pharmaceutical composition described herein above.

According to some embodiments, the present invention provides a method for treating or preventing development of cancer in a subject in need thereof, comprising administering to the subject a therapeutically effective amount of (a) chemotherapeutic agent and (b) a pharmaceutical composition described herein above.

According to some embodiments, there is provided a method for treating cancer in a subject being non-responsive to a chemotherapeutic agent, the method comprising administering to the subject a therapeutically effective amount of the chemotherapeutic agent and the pharmaceutical composition discloses herein.

In some embodiments, the method further comprises a step comprising selecting or identifying a subject as being non-responsive to a chemotherapeutic agent, and administering to the subject a therapeutically effective amount of the chemotherapeutic agent and the pharmaceutical composition discloses herein.

In some embodiments, the step comprising selecting or identifying is preceding the administering, proceeding the administering, or both, and administering to a subject selected or identified as being non-responsive to the chemotherapeutic agent a therapeutically effective amount of the chemotherapeutic agent and the pharmaceutical composition discloses herein.

In some embodiments, the non-responsive subject is characterized by expression of MAD2L2. In some embodiments, the non-responsive subject is characterized by an abnormal expression of MAD2L2. In some embodiments, the non-responsive subject is characterized by an over expression of MAD2L2. In some embodiments, the non-responsive subject is characterized by an expression or an over expression of one or more of: MAD2L2, Rev1, and MAD2L2-Rev1-complex. In some embodiments, the non-responsive subject is characterized by an over expression of MAD2L2-Rev1-complex.

In some embodiments, the subject is non-responsive to the chemotherapeutic agent, as disclosed herein. In some embodiments, the subject is non-responsive to one or more chemotherapeutic agents selected from: a crosslinking agent, a DNA complexing agent, a strand break agent, an alkylating agent, an anti-metabolite agent, a microtubule disruptor, a radiomimetic agent, a radiosensitizer, an intercalator, a DNA replication inhibitor, an anthracycline, an etoposide, and a topoisomerase II inhibitor, including any combination thereof.

In some embodiments, the subject is non-responsive to cisplatin. In some embodiments, the subject is non-responsive to a DNA damaging chemotherapeutic agent. In some embodiments, the chemotherapeutic agent is or comprises cisplatin.

In some embodiments, the chemotherapeutic agent, when combined with the pharmaceutical composition is administered at a dose at least 2 times, at least 5 times, at least 10 times, at least 15 times, at least 20 times, at least 30 times, at least 50 times, or at least 100 times lower, than the needed dose for the treatment of cancer using the chemotherapeutic agent without the pharmaceutical composition.

In some embodiments, the chemotherapeutic agent, when combined with the pharmaceutical composition is administered at a dose at least 2 times, at least 5 times, at least 10 times, at least 15 times, at least 20 times, at least 30 times, at least 50 times, or at least 100 times lower, than the needed dose for the prevention of development of cancer using the chemotherapeutic agent without the pharmaceutical composition.

In some embodiments, the pharmaceutical composition comprises a compound, pharmaceutically acceptable salt, isomer or tautomer thereof, wherein the compound is represented by Formula I:

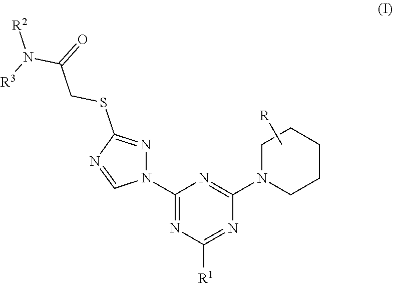

wherein: R, R¹, R² and R³ are independently selected from the group consisting of an optionally substituted heteroaryl, an optionally substituted aryl, a optionally substituted bicyclic aromatic ring, an optionally substituted aliphatic ring, an optionally substituted unsaturated aliphatic ring, an optionally substituted bicyclic aliphatic ring, an optionally substituted linear C₁-C₇ alkyl group, an optionally substituted branched C₁-C₇ alkyl group, an optionally substituted branched C₁-C₇ haloalkyl group, an optionally substituted linear C₁-C₇ haloalkyl group, an optionally substituted C₁-C₇ alkylhydroxy group, an optionally substituted C₁-C₇ alkoxy group, a hydrogen, a heteroatom, a hydroxy group, a halo group, an alkyl group, an alkoxy group, an amino group, or any combination thereof.

In some embodiments, the compound is:

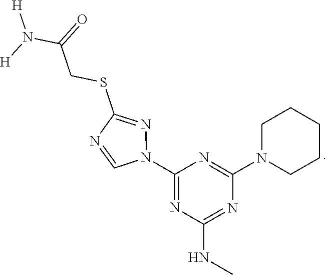 [SMILES: CNc1nc(N2CCCCC2)nc(-n2cnc(SCC(N)=O)n2)n1]

In some embodiments, the compound is:

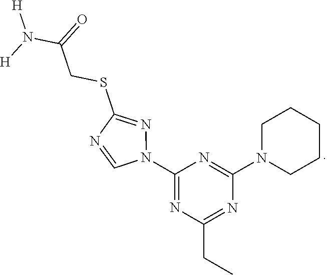 [SMILES: CCc1nc(N2CCCCC2)nc(-n2cnc(SCC(N)=O)n2)n1]

In some embodiments, the pharmaceutical composition comprises a compound, pharmaceutically acceptable salt, isomer or tautomer thereof, wherein the compound is represented by Formula II:

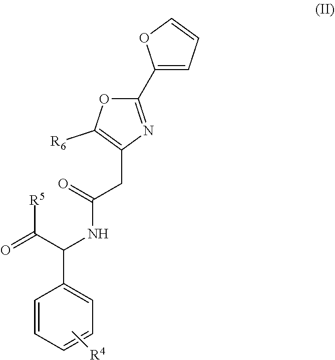

wherein: R⁴, R⁵ and R⁶ are independently selected from the group consisting of a heteroaryl, an aryl, a bicyclic aromatic ring, an aliphatic ring, an unsaturated aliphatic ring, a bicyclic aliphatic ring, a linear C₁-C₇ alkyl group, a branched C₁-C₇ alkyl group, a branched C₁-C₇ haloalkyl group, a linear C₁-C₇ haloalkyl group, a C₁-C₇ alkylhydroxy group, a C₁-C₇ alkoxy group, a hydrogen, a hydroxy group, a halo group, an alkyl group, an alkoxy group, an amino group, or any combination thereof.

In some embodiments, the compound is:

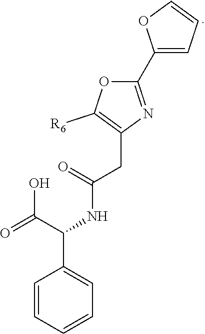

In some embodiments, the pharmaceutical composition comprises a compound, pharmaceutically acceptable salt, isomer or tautomer thereof, wherein the compound is represented by Formula III:

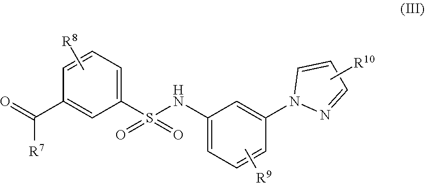

wherein: R⁷, R¹, R⁹ and R¹⁰ are independently selected from the group consisting of a heteroaryl, an aryl, a bicyclic aromatic ring, an aliphatic ring, an unsaturated aliphatic ring, a bicyclic aliphatic ring, a linear C₁-C₇ alkyl group, a branched C₁-C₇ alkyl group, a branched C₁-C₇ haloalkyl group, a linear C₁-C₇ haloalkyl group, a C₁-C₇ alkylhydroxy group, a C₁-C₇ alkoxy group, a hydrogen, a hydroxy group, a halo group, an alkyl group, an alkoxy group, an amino group, or any combination thereof.

In some embodiments, the compound is:

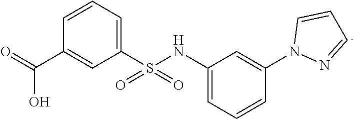 [SMILES: O=C(O)c1cccc(S(=O)(=O)Nc2cccc(-n3cccn3)c2)c1]

In some embodiments, the methods of the invention further comprise monitoring the at least one symptom selected from, without being limited thereto, growth or angiogenesis of a metastatic tumor, presence of circulating tumor cells, and appearance of a new metastasis.

In some embodiments, the methods of the invention further comprise monitoring the effect of the pharmaceutical composition on at least one symptom selected from, without being limited thereto, growth or angiogenesis of a metastatic tumor, presence of circulating tumor cells, and appearance of a new metastasis.

In some embodiments, the monitoring further comprises measuring a decrease of a symptom of the cancer or metastasis progression. In some embodiments, the symptom is selected from, without being limited thereto, size of a tumor, growth of a tumor, number of metastases, size of metastases, number of circulating tumor cells in blood, vascularization in tissue adjacent to the tumor, or metastases, vascularization of tumor or metastases, or, a weight loss of the subject. In some embodiment, the treating is inhibiting or reducing the proliferation of a cancer cell.

In some embodiments, the cancer is selected from the group consisting of lung cancer, breast cancer, osteosarcoma, neuroblastoma, colon adenocarcinoma, chronic myelogenous leukemia (CML), acute myeloid leukemia (AML), acute promyelocyte leukemia (APL), sarcoma, myxoma, rhabdomyoma, fibroma, lipoma, teratoma; bronchogenic carcinoma, alveolar carcinoma, bronchial adenoma, sarcoma, lymphoma, chondromatous hamartoma, mesothelioma, esophageal cancer, stomach cancer, pancreatic cancer, small bowel cancer, large bowel cancer; kidney cancer, bladder cancer, urethra cancer, prostate cancer, testis cancer; hepatoma, cholangiocarcinoma, hepatoblastoma, angiosarcoma, hepatocellular adenoma, hemangioma, osteogenic sarcoma, fibrosarcoma, malignant fibrous histiocytoma, chondrosarcoma, Ewing's sarcoma, malignant lymphoma, multiple myeloma, malignant giant cell tumor chordoma, osteochronfroma, benign chondroma, chondroblastoma, chondromyxofibroma, osteoid osteoma, giant cell tumors, cancer of the skuli, meninges cancer, brain cancer, spinal cord cancer, uterus cancer, cervical cancer, cancer of the ovaries, vulva cancer, vagina cancer, Hodgkin's disease, non-Hodgkin's lymphoma, malignant melanoma, basal cell carcinoma, squamous cell carcinoma, Karposi's sarcoma, moles dysplastic nevi, lipoma, angioma, and dermatofibroma.

In some embodiments, the chemotherapeutic agent is selected from the group consisting of doxorubicin, cisplatin, trans-platin, oxaliplatin, rituximab, hydroxyurea or trastuzumab.

### The Kit

According to some embodiments, the present invention provides a kit comprising: (a) a pharmaceutical composition described herein above; and (b) a chemotherapeutic agent.

In some embodiments, the kit comprises instructions for administering the pharmaceutical composition and the chemotherapeutic agent a w/w ratio ranging from 1:0.01 to 0.01:1, 1:0.09 to 0.01:1, 1:0.1 to 0.01:1, 1:0.5 to 0.01:1, 1:0.9 to 0.01:1, 1:1 to 0.01:1, 1:0.09 to 0.02:1, 1:0.1 to 0.02:1, 1:0.5 to 0.02:1, 1:0.9 to 0.02:1, 1:1 to 0.02:1, 1:0.09 to 0.05:1, 1:0.1 to 0.05:1, 1:0.5 to 0.05:1, 1:0.9 to 0.05:1, 1:1 to 0.05:1, 1:0.09 to 0.09:1, 1:0.1 to 0.09:1, 1:0.5 to 0.09:1, 1:0.9 to 0.09:1, 1:1 to 0.09:1, 1:0.09 to 0.1:1, 1:0.1 to 0.1:1, 1:0.5 to 0.1:1, 1:0.9 to 0.1:1, 1:1 to 0.1:1, 1:0.09 to 0.5:1, 1:0.1 to 0.5:1, 1:0.5 to 0.5:1, 1:0.9 to 0.5:1, or 1:1 to 0.5:1, including any range therebetween.

In some embodiments, administering is to a subject. In some embodiments, administering is to a cell culture.

In some embodiments, the kit comprises instructions for administering the pharmaceutical composition in a concentration ranging from 1 micromolar (μM) to 100 μM, 5 μM to 100 μM, 9 μM to 100 μM, 10 μM to 100 μM, 1 μM to 90 μM, 5 μM to 90 μM, 9 μM to 90 μM, 10 μM to 90 μM, 1 μM to 70 μM, 5 μM to 70 μM, 9 μM to 70 μM, 10 μM to 70 μM, 1 μM to 50 μM, 5 μM to 50 μM, 9 μM to 50 μM, or 10 μM to 50 μM, including any range therebetween.

In some embodiments, the present invention provides combined preparations. In one embodiment, “a combined preparation” defines especially a “kit of parts” in the sense that the combination partners as defined above can be dosed independently or by use of different fixed combinations with distinguished amounts of the combination partners i.e., simultaneously, concurrently, separately or sequentially. In some embodiments, the parts of the kit of parts can then, e.g., be administered simultaneously or chronologically staggered, that is at different time points and with equal or different time intervals for any part of the kit of parts. The ratio of the total amounts of the combination partners, in some embodiments, can be administered in the combined preparation. In one embodiment, the combined preparation can be varied, e.g., in order to cope with the needs of a patient subpopulation to be treated or the needs of the single patient which different needs can be due to a particular disease, severity of a disease, age, sex, or body weight as can be readily made by a person skilled in the art.

In one embodiment, depending on the severity and responsiveness of the condition to be treated, dosing can be of a single or a plurality of administrations, with course of treatment lasting from several days to several weeks or until cure is effected or diminution of the disease state is achieved.

In one embodiment, toxicity and therapeutic efficacy of the active ingredients described herein can be determined by standard pharmaceutical procedures in vitro, in cell cultures or experimental animals. In one embodiment, the data obtained from these in vitro and cell culture assays and animal studies can be used in formulating a range of dosage for use in human. In one embodiment, the dosages vary depending upon the dosage form employed and the route of administration utilized. In one embodiment, the exact formulation, route of administration and dosage can be chosen by the individual physician in view of the patient's condition. [See e.g., Fingl, et al., (1975) “The Pharmacological Basis of Therapeutics”, Ch. 1 p.1].

In one embodiment, compositions including the preparation of the present invention formulated in a compatible pharmaceutical carrier are prepared, placed in an appropriate container, and labeled for treatment of an indicated condition.

In one embodiment, compositions of the present invention are presented in a pack or dispenser device, such as an FDA approved kit, which contain one or more unit dosages forms containing the active ingredient. In one embodiment, the pack, for example, comprises metal or plastic foil, such as a blister pack. In one embodiment, the pack or dispenser device is accompanied by instructions for administration. In one embodiment, the pack or dispenser is accommodated by a notice associated with the container in a form prescribed by a governmental agency regulating the manufacture, use or sale of pharmaceuticals, which notice is reflective of approval by the agency of the form of the compositions or human or veterinary administration. Such notice, in one embodiment, is labeling approved by the U.S. Food and Drug Administration for prescription drugs or of an approved product insert.

The term “subject” as used herein refers to an animal, more particularly to non-human mammals and human organism. Non-human animal subjects may also include prenatal forms of animals, such as, e.g., embryos or fetuses. Non-limiting examples of non-human animals include: horse, cow, camel, goat, sheep, dog, cat, non-human primate, mouse, rat, rabbit, hamster, guinea pig, pig. In one embodiment, the subject is a human. Human subjects may also include fetuses. In one embodiment, a subject in need thereof is a subject afflicted with and/or at risk of being afflicted with a condition associated with increased bone resorption.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the term “prevention” of a disease, disorder, or condition encompasses the delay, prevention, suppression, or inhibition of the onset of a disease, disorder, or condition. As used in accordance with the presently described subject matter, the term “prevention” relates to a process of prophylaxis in which a subject is exposed to the presently described peptides prior to the induction or onset of the disease/disorder process. This could be done where an individual has a genetic pedigree indicating a predisposition toward occurrence of the disease/disorder to be prevented. For example, this might be true of an individual whose ancestors show a predisposition toward certain types of, for example, inflammatory disorders. The term “suppression” is used to describe a condition wherein the disease/disorder process has already begun but obvious symptoms of the condition have yet to be realized. Thus, the cells of an individual may have the disease/disorder but no outside signs of the disease/disorder have yet been clinically recognized. In either case, the term prophylaxis can be applied to encompass both prevention and suppression. Conversely, the term “treatment” refers to the clinical application of active agents to combat an already existing condition whose clinical presentation has already been realized in a patient.

As used herein, the term “condition” includes anatomic and physiological deviations from the normal that constitute an impairment of the normal state of the living animal or one of its parts, that interrupts or modifies the performance of the bodily functions.

Any concentration ranges, percentage range, or ratio range recited herein are to be understood to include concentrations, percentages or ratios of any integer within that range and fractions thereof, such as one tenth and one hundredth of an integer, unless otherwise indicated.

As used herein, the terms “subject” or “individual” or “animal” or “patient” or “mammal,” refers to any subject, particularly a mammalian subject, for whom therapy is desired, for example, a human.

### Pharmaceutically Acceptable Salts

In some embodiments, the compounds of the present invention can exist in free form for treatment, or as a pharmaceutically acceptable salt.

As used herein, the term “pharmaceutically acceptable salt” refers to any non-toxic salt of a compound of the present invention that, upon administration to a subject, e.g., a human, is capable of providing, either directly or indirectly, a compound of this invention or an inhibitorily active metabolite or residue thereof. For example, the term “pharmaceutically acceptable” can mean approved by a regulatory agency of the Federal or a state government or listed in the U. S. Pharmacopeia or other generally recognized pharmacopeia for use in animals, and more particularly in humans.

As used herein, the term “carrier” refers to a diluent, adjuvant, excipient, or vehicle with which the therapeutic compound is administered. Such pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerin, propylene glycol or other synthetic solvents. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; and agents for the adjustment of tonicity such as sodium chloride or dextrose are also envisioned. The carrier may comprise, in total, from about 0.1% to about 99.99999% by weight of the pharmaceutical compositions presented herein.

Pharmaceutically acceptable salts are well known in the art. For example, S. M. Berge et al., describe pharmaceutically acceptable salts in detail in J. Pharmaceutical Sciences, 1977, 66, 1-19. Pharmaceutically acceptable salts of the compounds of this invention include those derived from suitable inorganic and organic acids and bases. These salts can be prepared in situ during the final isolation and purification of the compounds. Acid addition salts can be prepared by 1) reacting the purified compound in its free-based form with a suitable organic or inorganic acid and 2) isolating the salt thus formed.

Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, valerate salts, and the like.

Base addition salts can be prepared by 1) reacting the purified compound in its acid form with a suitable organic or inorganic base and 2) isolating the salt thus formed. Salts derived from appropriate bases include alkali metal (e.g., sodium, lithium, and potassium), alkaline earth metal (e.g., magnesium and calcium), ammonium and N+(C1-4alkyl)4 salts. This invention also envisions the quaternization of any basic nitrogen-containing groups of the compounds disclosed herein. Water or oil-soluble or dispersible products may be obtained by such quaternization.

Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate. Other acids and bases, while not in themselves pharmaceutically acceptable, may be employed in the preparation of salts useful as intermediates in obtaining the compounds of the invention and their pharmaceutically acceptable acid or base addition salts.

In some embodiments, the compounds described herein are chiral compounds (i.e. possess an asymmetric carbon atom). In some embodiments, diastereomers, geometric isomers and individual isomers are encompassed within the scope of the present invention. In some embodiments, a chiral compound described herein is in form of a racemic mixture. In some embodiments, a chiral compound is in form of a single enantiomer, with an asymmetric carbon atom having the R configuration. In some embodiments, a chiral compound is in form of a single enantiomer, with an asymmetric carbon atom having the S configuration as described hereinabove.

In some embodiments, a chiral compound is in form of a single enantiomer with enantiomeric purity of more than 70%. In some embodiments, a chiral compound is in form of a single enantiomer with enantiomeric purity of more than 80%. In some embodiments, a chiral compound is in form of a single enantiomer with enantiomeric purity of more than 90%. In some embodiments, a chiral compound is in form of a single enantiomer with enantiomeric purity of more than 95%.

In some embodiments, the compound of the invention comprising an unsaturated bond is in a form of a trans-, or cis-isomer. In some embodiments, the composition of the invention comprises a mixture of cis- and trans-isomers, as described hereinabove.

In some embodiments, the compounds described herein can exist in unsolvated form as well as in solvated form, including hydrated form. In general, the solvated form is equivalent to the unsolvated form and is encompassed within the scope of the present invention. Certain compounds of the present invention may exist in multiple crystalline or amorphous forms. In general, all physical forms are equivalent for the uses contemplated by the present invention and are intended to be within the scope of the present invention.

The term “solvate” refers to a complex of variable stoichiometry (e.g., di-, tri-, tetra-, penta-, hexa-, and so on), which is formed by a solute (the conjugate described herein) and a solvent, whereby the solvent does not interfere with the biological activity of the solute. Suitable solvents include, for example, ethanol, acetic acid and the like.

The term “hydrate” refers to a solvate, as defined hereinabove, where the solvent is water.

Unless otherwise indicated, structures depicted herein are also meant to include all isomeric (e.g., enantiomeric, diastereomeric, geometric, conformational, and rotational) forms of the structure. For example, the R and S configurations for each asymmetric center, (Z) and (E) double bond isomers, and (Z) and (E) conformational isomers are included in this invention. As would be understood to one skilled in the art, a substituent can freely rotate around any rotatable bonds. Therefore, single stereochemical isomers as well as enantiomeric, diastereomeric, geometric, conformational, and rotational mixtures of the present compounds are within the scope of the invention.

Unless otherwise indicated, all tautomeric forms of the compounds of the invention are within the scope of the invention.

Additionally, unless otherwise indicated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays.

In another embodiment, the compositions of the invention take the form of solutions, suspensions, emulsions, tablets, pills, capsules, powders, gels, creams, ointments, foams, pastes, sustained-release formulations and the like. In another embodiment, the compositions of the invention can be formulated as a suppository, with traditional binders and carriers such as triglycerides, microcrystalline cellulose, gum tragacanth or gelatin. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in: Remington's Pharmaceutical Sciences” by E. W. Martin, the contents of which are hereby incorporated by reference herein. Such compositions will contain a therapeutically effective amount of the polypeptide of the invention, preferably in a substantially purified form, together with a suitable amount of carrier so as to provide the form for proper administration to the subject.

### Definitions

As used herein, the term “alkyl” describes an aliphatic hydrocarbon including straight chain and branched chain groups. Preferably, the alkyl group has 20 or less main-chain carbons. The alkyl can be substituted or unsubstituted, as defined herein.

The term “alkyl”, as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” describes an all-carbon monocyclic or fused ring (i.e. rings which share an adjacent pair of carbon atoms) group where one or more of the rings does not have a completely conjugated pi-electron system. The cycloalkyl group may be substituted or unsubstituted, as indicated herein.

The term “aryl” describes an all-carbon monocyclic or fused-ring polycyclic (i.e. rings which share adjacent pairs of carbon atoms) groups having a completely conjugated pi-electron system. The aryl group may be substituted or unsubstituted, as indicated herein.

The term “alkoxy” describes both an O-alkyl and an —O-cycloalkyl group, as defined herein.

The term “aryloxy” describes an —O-aryl, as defined herein.

Each of the alkyl, cycloalkyl and aryl groups in the general formulas herein may be substituted by one or more substituents, whereby each substituent group can independently be, for example, halide, alkyl, alkoxy, cycloalkyl, alkoxy, nitro, amino, hydroxyl, thiol, thioalkoxy, thiohydroxy, carboxy, amide, aryl and aryloxy, depending on the substituted group and its position in the molecule. Additional substituents are also contemplated.

The term “haloalkoxy” describes an alkoxy group as defined herein, further substituted by one or more halide(s).

The term “hydroxyl” or “hydroxy” describes a —OH group.

The term “mercapto” or “thiol” describes a —SH group.

The term “thioalkoxy” describes both an —S-alkyl group, and a —S-cycloalkyl group, as defined herein.

The term “thioaryloxy” describes both an —S-aryl and a —S-heteroaryl group, as defined herein.

The term “amino” describes a —NR′R″ group, with R′ and R″ as described herein.

The term “heteroalicyclic” or “heterocyclyl” describes a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydrofurane, tetrahydropyrane, morpholino and the like.

The term “carboxy” or “carboxylate” describes a —C(O)OR′ group, where R′ is hydrogen, alkyl, cycloalkyl, alkenyl, aryl, heteroaryl (bonded through a ring carbon) or heteroalicyclic (bonded through a ring carbon) as defined herein.

The term “carbonyl” or “keto” describes a —C(O)R′ group, where R′ is as defined hereinabove.

The above-terms also encompass thio-derivatives thereof (thiocarboxy and thiocarbonyl).

The term “thiocarbonyl” describes a —C(S)R′ group, where R′ is as defined hereinabove.

A “thiocarboxy” group describes a —C(S)OR′ group, where R′ is as defined herein.

A “sulfinyl” group describes an —S(O)R′ group, where R′ is as defined herein.

A “sulfonyl” or “sulfonate” group describes an —S(O)₃R′ group, where R′ is as defined herein.

A “sulfone” group describes an —S(O)₂R′ group, where R′ is as defined herein.

A “carbamyl” or “carbamate” group describes an —OC(O)NR′R″ group, where R′ is as defined herein and R″ is as defined for R′.

A “nitro” group refers to a —NO₂ group.

The term “amide” as used herein encompasses C-amide and N-amide.

The term “C-amide” describes a —C(O)NR′R″ end group or a —C(O)NR′-linking group, as these phrases are defined hereinabove, where R′ and R″ are as defined herein.

The term “N-amide” describes a —NR″C(O)R′ end group or a —NR′C(O)— linking group, as these phrases are defined hereinabove, where R′ and R″ are as defined herein.

The term “carboxylic acid derivative” as used herein encompasses carboxy, amide, carbonyl, anhydride, carbonate ester, and carbamate.

A “cyano” or “nitrile” group refers to a —CN group.

The term “azo” or “diazo” describes an —N═NR′ end group or an —N═N— linking group, as these phrases are defined hereinabove, with R′ as defined hereinabove.

The term “guanidine” describes a —R′NC(N)NR″R″′ end group or a —R′NC(N) NR″-linking group, as these phrases are defined hereinabove, where R′, R″ and R′ are as defined herein.

As used herein, the term “azide” refers to a —N₃ group.

The term “sulfonamide” refers to a —S(O)₂NR′R″ group, with R′ and R″ as defined herein.

The term “phosphonyl” or “phosphonate” describes an —OP(O)—(OR′)₂ group, with R′ as defined hereinabove.

The term “phosphinyl” describes a —PR′R″ group, with R′ and R″ as defined hereinabove.

The term “alkylaryl” describes an alkyl, as defined herein, which substituted by an aryl, as described herein. An exemplary alkylaryl is benzyl.

The term “heteroaryl” describes a monocyclic or fused ring (i.e. rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted by one or more substituents, as described hereinabove. Representative examples are thiadiazole, pyridine, pyrrole, oxazole, indole, purine and the like.

The term “halide”, or “halo” describes fluorine, chlorine, bromine or iodine.

The term “haloalkyl” describes an alkyl group as defined above, further substituted by one or more halide(s).

The term “vinyl” refers to a —CH═CH₂ group.

The term “allyl” refers to a —CH₂—CH═CH₂ group.

The term “thioalkyl” describes an alkyl group as defined above, further substituted by one or more mercapto group(s).

The term “alkylhydroxy” describes an alkyl group as defined above, further substituted by one or more hydroxy group(s).

### General

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Methods

### Colony Survival

H1975 cells were plated in 24-well plates at 50,000 cells/well (for 50 M inhibitor treatment) or 20,000 cells/well (for 10 M inhibitor treatment). U2OS and A549 cells were plated in 24-well plates at 5000 cells/well. The next day, cells were treated with varying concentrations of cisplatin, either with or without added inhibitor compound. After 48 hours of cisplatin+compound treatment, the cells were washed 3 times with PBS×1 and fresh media applied. After 4 days of recovery, the cells were stained with methylene blue and imaged using a Nikon SMZ25 stereomicroscope. The images were analyzed using the ImageJ (National Institutes of Health) area measurement tool to quantify total colony size in each well.

### GFP-Trap A Immunoprecipitation (IP)

HEK293 cells were plated in 10-cm plates at 40-50% confluency. The next day, the cells were co-transfected with YFP-Rev1 and myc-MAD2L2 plasmids. After 24 hours, 50 M inhibitor compounds (or DMSO negative control) were applied to the cells and growth continued for another 24 hours. The cells were then harvested and protein extraction was performed, followed by IP pulldown of the YFP-Rev1 species with GFP-Trap A conjugated agarose beads, to assess the levels of Co-IP'ed myc-REV7.

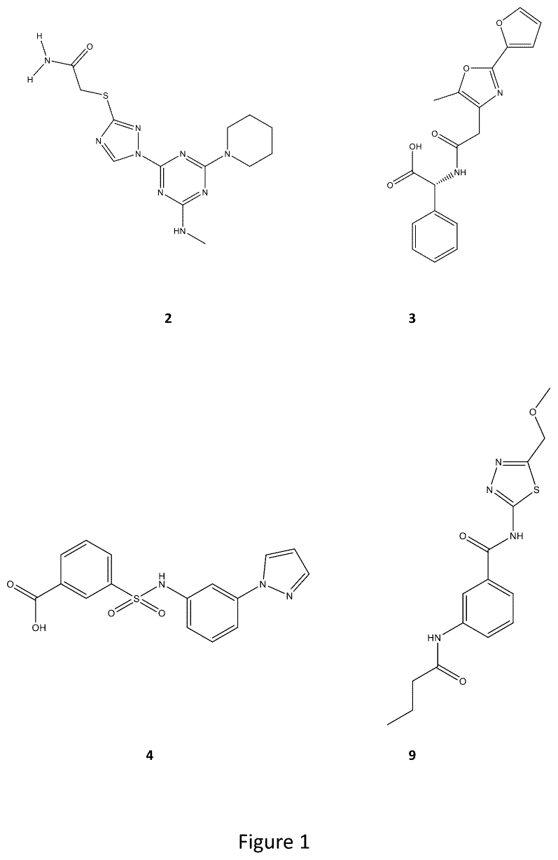

Chemical structure of inhibitor compounds 2, 3, 4 and 9 used in the cell survival and Co-IP assays are presented in FIG. 1.

### Generating MAD2L2-MAD2L2-Rev1 Model

The dimeric form of the structure of MAD2L2 in complex with a CAMP fragment was built from the monomeric structure (PDB code 5XPT) using the crystal symmetry matrix as shown in the above paper. Rev1 was added to one of the MAD2L2 monomers by superimposition of the MAD2L2-Rev1 complex structure (PDB 3VU7). The position of Rev1 was further refined by performing local docking of Rev1 to the MAD2L2 dimer using the Rosetta Online Server. The top five docking results were examined and three of them were similar. The middle pose of the three similar results was selected for molecular dynamics simulation and missing amino acids in the structure were added using Modeller. Molecular dynamics simulations were carried out using GROMACS software using the AMBER36 force-field and TIP3P water model, with an ionic strength of 100 mmol/L NaCl. The system was backbone restrained and energy minimized, followed by a two-step equilibration process of 100 ps to the target temperature and pressure of 310 K and 1 bar. Backbone restraints were lifted, and a 30 ns long full MD simulation with a 2-fs time step under constant pressure and temperature was conducted. Snapshots were taken every 0.1 ns, the last 10 frames were compared by RMSD and the conformation that showed the highest similarity to the other frames was selected for docking simulations.

### Docking Simulations

High throughput virtual screening virtual docking of small molecules was carried out in the cavity of the MAD2L2 homodimer interface of the above conformation after removing Rev1. Docking simulations were done using the University of California at San Francisco, San Francisco, CA (UCSF Dock; v6.8) with ZINC12 Clean Leads subset of small ligands (Subset ID 11). This dataset contains 4,591,276 molecules selected according to the following criteria: p.mwt<=350 and p.mwt>=250 and p.xlogp<=3.5 and p.rb<=7. First, all molecules were docked using the flex anchor-and-grow (fast) method for rigid receptor and flexible ligand with grid-based scoring. After an initial large-scale scanning, the best 5000 compounds were docked and rescored using AMBER force files with default scoring parameters allowing flexibility for the ligand and receptor amino acids with a cut-off distance of 2.5 Å from the ligand. Molecules making less than 2 Hb with the MAD2L2 homodimer or having an Amber_Score binding energy of less than −10 were filtered out. The final selection step was done via manual selection, in which the remaining candidate ligand-protein complexes were visually scanned and ranked from 1 (compound binding at residues not essential for MAD2L2-Rev1 binding) to 4 (compound binding tightly at residues essential for MAD2L2-Rev1 binding). Specifically, the compounds predicted by visual estimation to bind most closely to the K1203 and L1205 interface residues of Rev1 (essential MAD2L2 binding residues) were ranked highest (4). Of the compounds ranked as 4, the 10 compounds available for purchase from suppliers were selected for experimental validation.

### Protein Induction, Purification and Field Effect Biosensing (FEB)

E. coli BL21 Star cells were transfected with GST-MAD2L2 vector. Protein induction was done for 4 hours in 30° C., with 1 mM IPTG. Purification was done according to standard GST protocol.

The binding kinetics of MAD2L2 to the small molecule compounds were measured by the Field Effect Biosensing (FEB) Agile R100 label-free binding assay (Nanomedical Diagnostics Inc), following their standard protocol. Briefly, 500 nM of purified MAD2L2 was immobilized on a graphene sensor chip through amine groups. The current baseline level for the chip was recorded in PBS. Next, PBS was aspirated, and the changes in the baseline current induced by 50 μl of 1, 10, 20, 30, 40 and 50 μM droplets of the tested compound were recorded. K_{D} values were calculated by the DataLine 2.0 software by either a Hill equation fit or by using the kon and koff values at a single concentration. The K_{D} values obtained by these two methods were almost identical.

### Cell Culture and Transfections

The A549, HEK293, and 293T cells were cultured in Dulbecco's Modified Eagle

Medium/DMEM (Biological Industries Israel Beit Haemek; 01-052-1A) with 4.5 g/l D-glucose, 4 mM L-glutamine, 10% fetal bovine serum (Biological Industries Israel Beit Haemek; 04-007-1A), and 1% penicillin/streptomycin. The H1975 cells were cultured in RPMI 1640 medium (Biological Industries Israel Beit Haemek; 01-100-1A) with 4.5 g/l D-glucose, 4 mM L-glutamine, 10% fetal bovine serum (Biological Industries Israel Beit Haemek; 04-007-1A), and 1% penicillin/streptomycin. Transfections of HEK293 cells with plasmid DNA were performed using the Avalanche® Everyday Transfection Reagent (EZT-EVDY-1); according to the manufacturer's protocol. Briefly, cells were passaged the day before transfection to reach a confluency of 60-70%. The next day, the selected plasmid DNA was incubated in serum-free media with the recommended volume of transfection reagent for 20 minutes at room temperature. This transfection mix was gently added to the prepared cell culture plate(s) for continued incubation at 37° C. For co-IP experiment plates, after 24 hours, the relevant compound (DMSO vehicle, c #2, or c #3) was added in pre-warmed media, and the transfected plates were incubated for another 24 hours at 37° C. until harvesting.

### Colony Survival Assay

Cells were plated in 24-well plates at 50,000 cells/well (H1975 line) or 10,000 cells/well (A549 line). The next day, cells were treated with varying concentrations of cisplatin (0-10 M), with or without 50 μM compound. For the untreated control wells containing only cisplatin, DMSO was applied in place of the compound. After 48 hours of cisplatin+compound treatment, the cells were washed 3 times with PBS and fresh media was applied. After 4 days of recovery, the cells were stained with methylene blue and imaged using a Nikon SMZ25 stereomicroscope. The images were analyzed using the ImageJ (National Institutes of Health) area measurement tool to quantify total colony size in each well.

### Western Blot and Co-Immunoprecipitation

HEK293 cells were harvested 48 h following co-transfection and lysed in extraction buffer (50 mM Tris pH 8, 150 mM NaCl, 20 mM EDTA, 50 mM NaF, 1% TritonX) supplemented with Merck® 1000×protease inhibitor (539134). Cells were lysed on ice for 30 minutes and centrifuged at 20,000 g for 30 minutes at 4° C. For immunoblotting, extracts were boiled in Laemmli buffer for 5 min. Equal amounts of protein sample (30 μg) were loaded on 8%-12% acrylamide gel and transferred to a nitrocellulose membrane (Amersham). For immunoprecipitations, clarified lysates were supplemented with 7 μl of equilibrated GFP-Trap antibody-conjugated agarose beads (Chromotek; gta-20) and incubated for 1-2 h at 4° C. Beads were washed three times in PBS buffer and boiled in Laemmli buffer for 5 minutes. The following primary antibodies were used for immunoblotting: Myc (Santa Cruz; SC-40) 1:1000 dilution, GFP (Santa Cruz, SC-9996) 1:1000 dilution, MAD2L2 (ProteinTech; 12683-1-AP). Appropriate light-chain specific secondary antibodies were used at 1:10,000 for all membranes: anti-mouse (Jackson ImmunoResearch Laboratories, Inc; 115-035-174) anti-rabbit (EMD Millipore; MAB201P). The membranes were developed with the BioRad biomolecular imager, and band densitometry was performed using the ImageJ (National Institutes of Health) Gel Analyzer function. All statistical analysis was performed with Prism 9.1.2 (GraphPad Software).

### Immunofluorescence

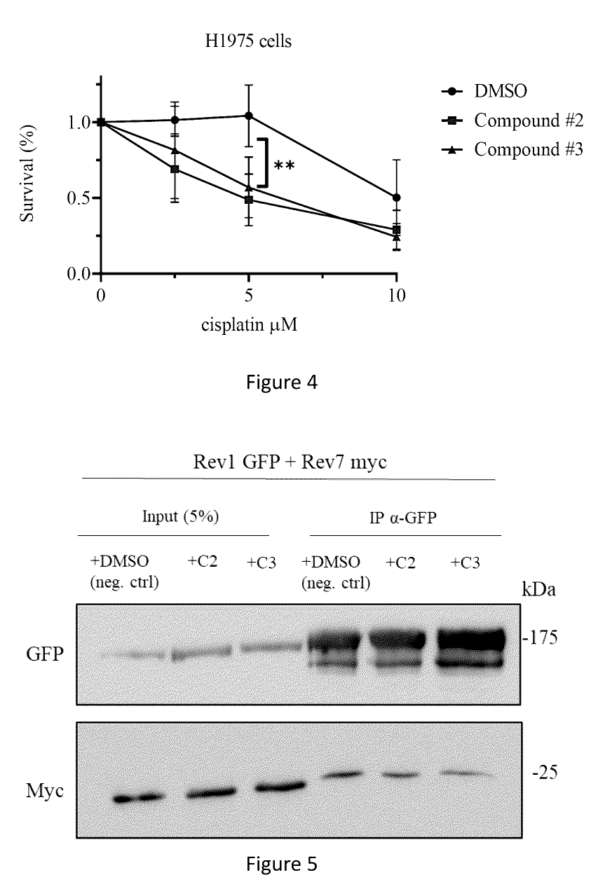

H1975 and A549 cells were grown on glass coverslips in 12-well plates with the appropriate media, and fixed in 4% paraformaldehyde for 10 minutes at room temperature. Cells were permeabilizedin 0.5% TritonX100 in PBS×1 for 10 minutes at room temperature and then blocked in 5% BSA in 0.1% PBS×1-Tween for 1 hour at room temperature. Anti-phospho-Histone H2A.X primary antibody (Mercury; 05-636-25UG) diluted 1:400 in 5% BSA in 0.1% PBS×1-Tween was added for 1 hour at room temperature. Fluorescent-dye conjugated secondary antibody was applied for 1 hour at room temperature. The coverslips were washed between each step with PBS×1. Nuclei were stained with DAPI (1:2000 dilution) at room temperature in the dark for 3 minutes. Coverslips were mounted on glass slides and imaged using an Olympus 1×81 microscope. For all treatments, cells with fewer than 80 foci were scored manually (FIG. 4B), and cells with more than 80 foci and very bright signals were scored automatically by using the “Analyze Particles” function in ImageJ (National Institutes of Health) after setting a suitable threshold.

### Cellular Thermal Shift Assay (CETSA)

The in vivo binding of MAD2L2 to the small molecule compounds was detected using the cellular thermal shift assay (CETSA), following their standard protocol. Briefly, 293T cells were grown to confluency and treated for 1 hour with the DMSO or selected compounds at 50 M. Cells were collected and centrifuged for 3 min at 300 g, washed once with PBS, and each pellet was resuspended in 1 ml of PBS supplemented with Merck® 1000×protease inhibitor (539134). The cell suspensions were aliquoted and incubated for 3 min in the VWR XT96 thermal cycler using a gradient from 40°−55° C. Following heating, the tubes were incubated at room temperature for 3 min and then snap frozen in liquid nitrogen. The samples were then lysed via two subsequent freeze-thaw cycles with vortexing, and the lysates were centrifuged at 20,000 g for 20 min at 4° C. Finally, the cleared lysates were boiled in Laemmli buffer for 5 minutes.

### Example 1

### Colony Survival Assay

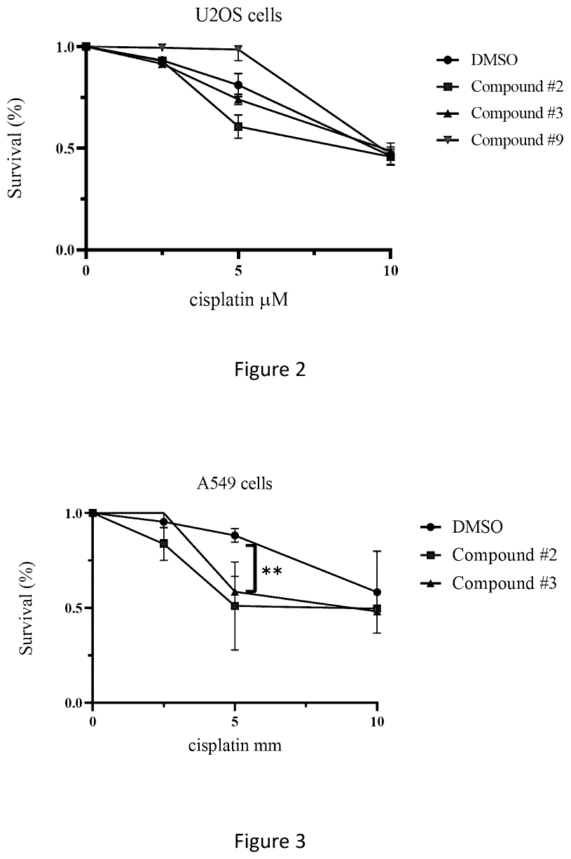

FIG. 2 presents a graph of colony survival assay on U2OS cells treated with varying concentrations of cisplatin without added inhibitor (DMSO), with compound 2 as inhibitor (Compound #2), with compound 3 as inhibitor (Compound #3) and with compound 9 as inhibitor (Compound #9).

FIG. 3 presents a graph of colony survival assay on A549 cells treated with varying concentrations of cisplatin without added inhibitor (DMSO), with compound 2 as inhibitor (Compound #2) and with compound 3 as inhibitor (Compound #3).

FIG. 4 presents a graph of colony survival assay on H1975 cells treated with varying concentrations of cisplatin without added inhibitor (DMSO), with compound 2 as inhibitor (Compound #2) and with compound 3 as inhibitor (Compound #3).

Both compounds 2 and 3 showed reduction in cell survival, while compound 9 and DMSO (negative control) showed no activity.

### Example 2

### GFP-Trap a Immunoprecipitation (IP) Assay

10 cm plates of HEK293 cells were transfected with Rev1 YFP (3 μg) and Rev7 myc (3 μg). After 24 hours, compounds (2, 3, or DMSO negative control) were added at 50 μM concentration in prewarmed media. Cells were replated in 6-well plates (2 wells per sample/compound). The next day, anti-GFP IP was performed. The cells treated with compound 2 and compound 3 showed reduction in Rev1-Rev7 binding interaction (quantification shown above in table, normalized either to IP efficiency or to co-IP input levels (FIG. 5).

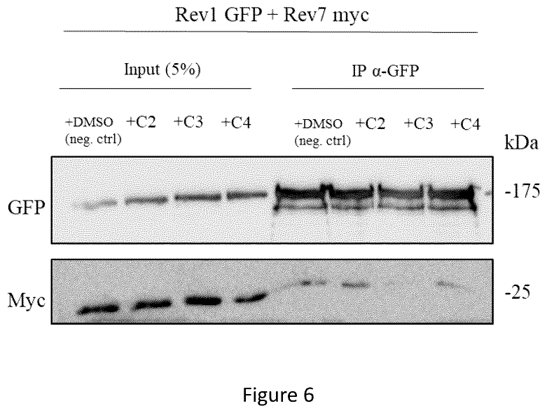

10 cm plates of HEK HEK293 cells were transfected with Rev1 YFP (3 μg) and Rev7 myc (3 μg). After 24 hours, compounds (2, 3, 4, or DMSO negative control) were added at 50 μM concentration in prewarmed media. Cells were replated in 6-well plates (2 wells per sample/compound). The next day, anti-GFP IP was performed. The cells treated with compound 2 and compound 3 showed reduction in Rev1-Rev7 binding interaction (quantification shown above in table, normalized either to IP efficiency or to co-IP input levels (FIG. 6).

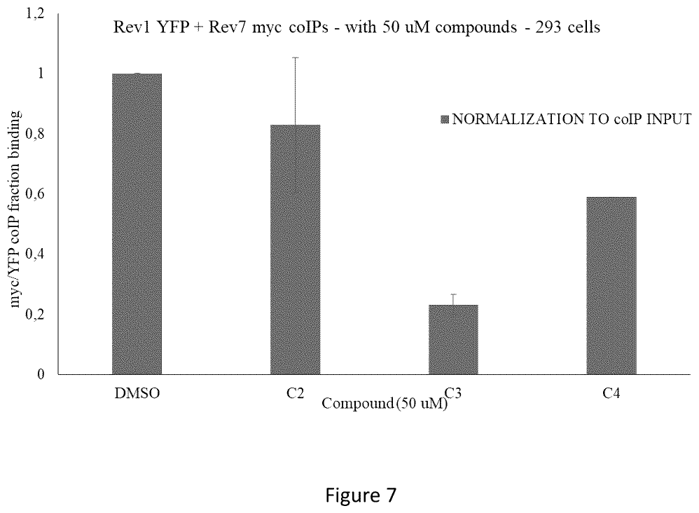

FIG. 7 presents a graphical summary of results from two co IP experiments in HEK293 cells, with 50 μM applied compounds. Error bars show standard deviation. For DMSO, C₂, and C₃, n=2. For C₄, n=1.

### Example 3

### MAD2L2-MAD2L2-Rev1 Model and Docking Simulations

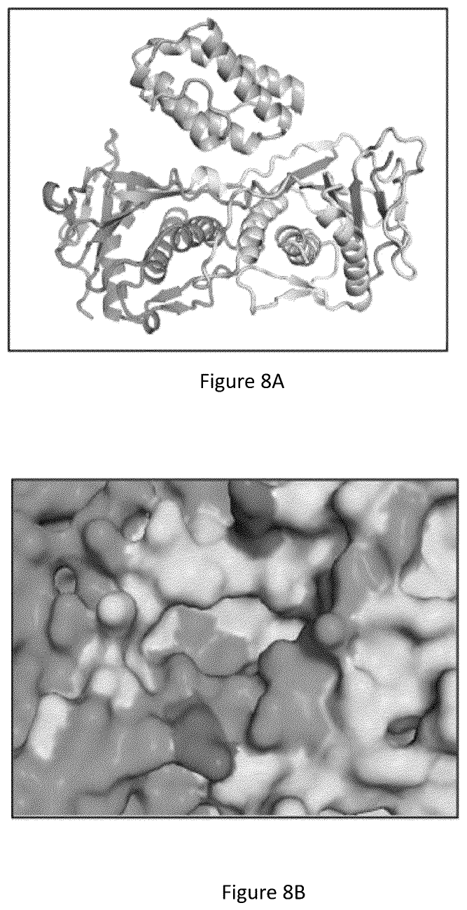

The dimeric form of MAD2L2 in complex with a CAMP fragment and Rev1 was built as described in Methods. The complex was placed in a box of water and a 30 ns long molecular dynamics simulation (MD) was conducted to determine the structural changes that occur upon binding of Rev1 to the MAD2L2 dimer. A representative conformation (see Methods) from the last Ins was selected (FIG. 8A). Most of the interactions between Rev1 and the MAD2L2 dimer are at residues L186-K198 of one of the MAD2L2 monomers and K198-Y202 of the other monomer. K190 in one of the residues important for MAD2L2 dimerization. Thus, despite the 2-fold symmetry of the MAD2L2 dimer, Rev1 binds to it asymmetrically. During the MD simulation, the cavity between the two MAD2L2 proteins became slightly wider. Binding of Rev1 results in an induced fit of the MAD2L2 dimer. The Cα-Cα distance between K190 of the two monomers increases from 15.86 to 18.10 Å, and the sidechains undergo reorientation for optimal binding of MAD2L2 (FIG. 8B).

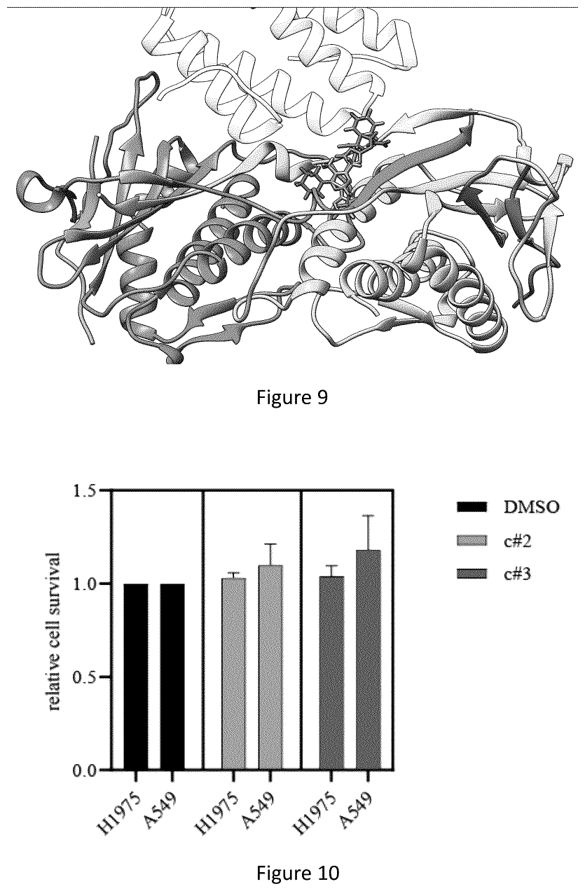

High-throughput virtual screening was conducted using DOCK6 software with ZINC12 Clean Leads subset to identify potential lead compounds that bind to the reshaped cavity. Docking was done in two stages. Initially, all molecules were docked to the cavity using the fast anchor-and-grow algorithm with rigid receptor and flexible ligand. The top 5000 ranked compounds were docked and rescored using AMBER force files with default scoring parameters allowing flexibility for the ligand and receptor amino acids contacting the ligand (see Methods). The best ranking ligands were manually selected based on their ability to interfere with the interaction of Rev1 with the MAD2L2 dimer. Representative docking of two selected compounds ZINC97017995 (c #2) and ZINC25496030 (c #3) in the MAD2L2 dimer cavity, is presented in FIG. 9. Two-dimensional drawings of c #2 and c #3 are presented in FIGS. 2 and 3, respectively.

### Example 4

### Compound c #2 and Compound c #3 Sensitize Cells to Cisplatin

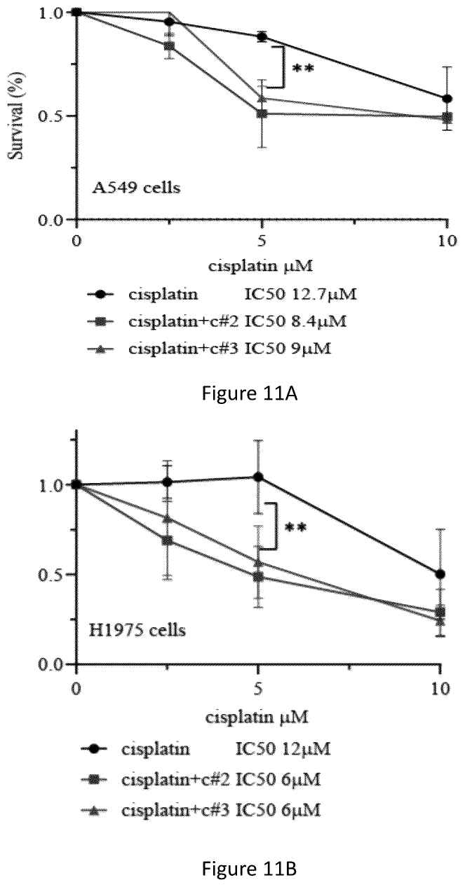

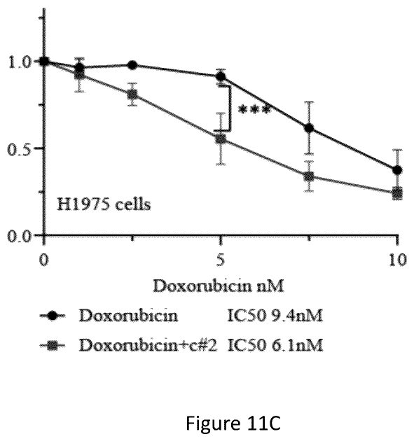

In order to assess the ability of the selected compounds to sensitize cells during cisplatin treatment, the inventors established a colony survival assay comparing the viability of cells treated with cisplatin alone or with combined treatment of cisplatin and 50 μM compound. For the compound screen, we used two lung cancer cell lines: human non-small lung carcinoma H1975 cells, and epithelial lung adenocarcinoma A549 cells. Out of ten compounds tested, compounds c #2 and c #3 were identified with the most significant sensitization effect and were chosen for future characterization. Overall, treatment with c #2 or c #3 alone caused no toxicity during the colony survival assay (FIG. 10). However, when cells were exposed to a combined treatment of cisplatin and 50 μM of the compound, an approximately twofold sensitization effect was observed. Survival rate was reduced from 90% in 5 μM cisplatin treatment to 50% survival in the combined treatment (FIGS. 11A-B). H1975 cells presented similar behavior following doxorubicin treatment (FIG. 11C). IC50 was calculated using linear regression, presenting a clear reduction of cisplatin's IC50 in all treatments. These findings suggest that combined treatment with c #2 or c #3 is efficient in increasing cell death at low cisplatin concentrations, potentially allowing use of lower doses of cisplatin in future cancer treatment.

### Example 5

DNA Damage is Elevated after Co-Treatment of Cisplatin Together with c #2 or c #3

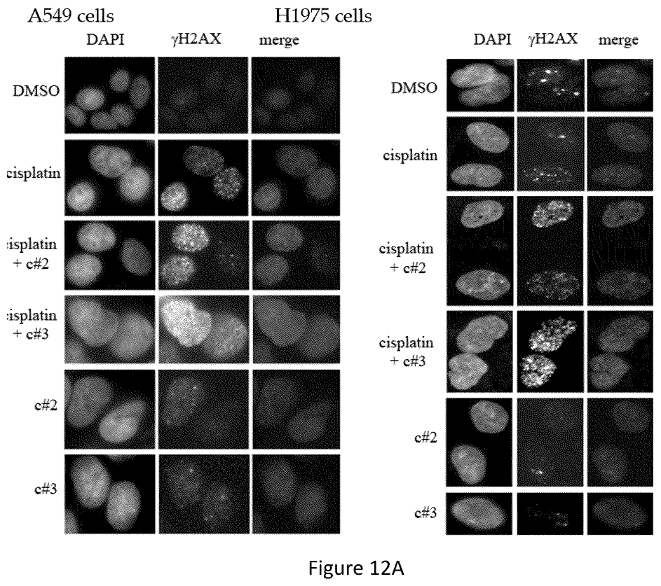

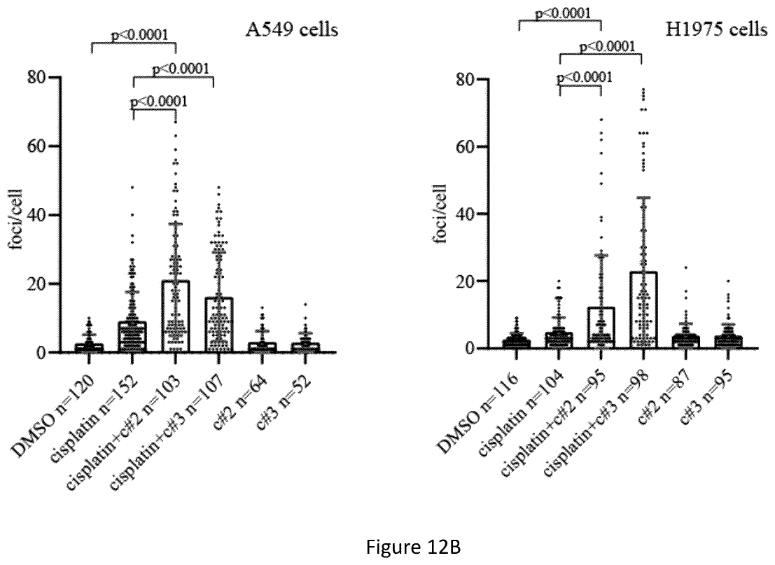

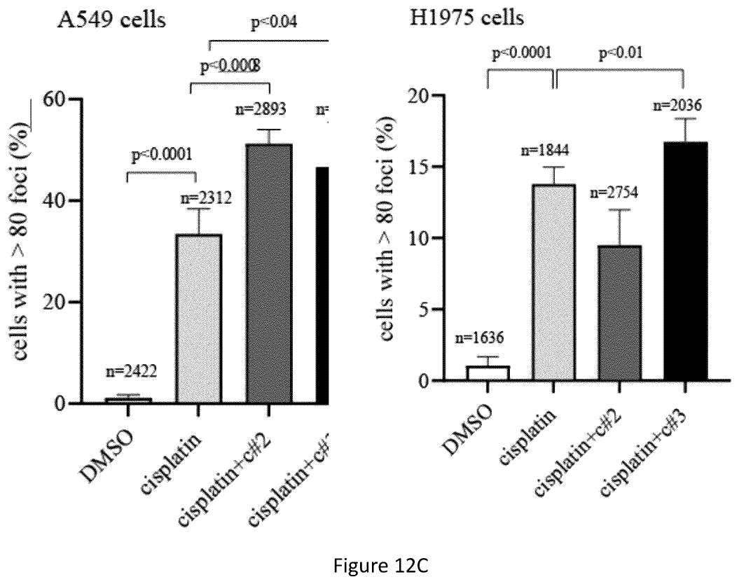

Next, the inventors examined whether combined treatment of cisplatin with c #2 or c #3 correlates with an increase in DNA damage, which might explain the compound's sensitization effect. A549 and H1975 cell lines were exposed to combined overnight treatment with cisplatin and c #2 or c #3, and the levels of γH2AX foci were monitored. In both cell lines, a clear increase in the number of foci was observed after combined treatment, compared to cells treated with cisplatin or compounds alone (FIG. 12A). After quantification, we observed a significant increase in the number of γH2AX foci/cell in the combined treatment (FIG. 12B; A549 left panel, H1975 right panel). Moreover, cells with more than 80 foci or very bright signal were significantly more abundant in the combined treatment (FIG. 12C; A549 left panel, H1975 right panel). The results show that enhanced cell death after cisplatin treatment in the presence of the compound is associated with increased DNA damage.

### Example 6

MAD2L2-Rev1 Interaction is Reduced after Exposure to c #2 or c #3

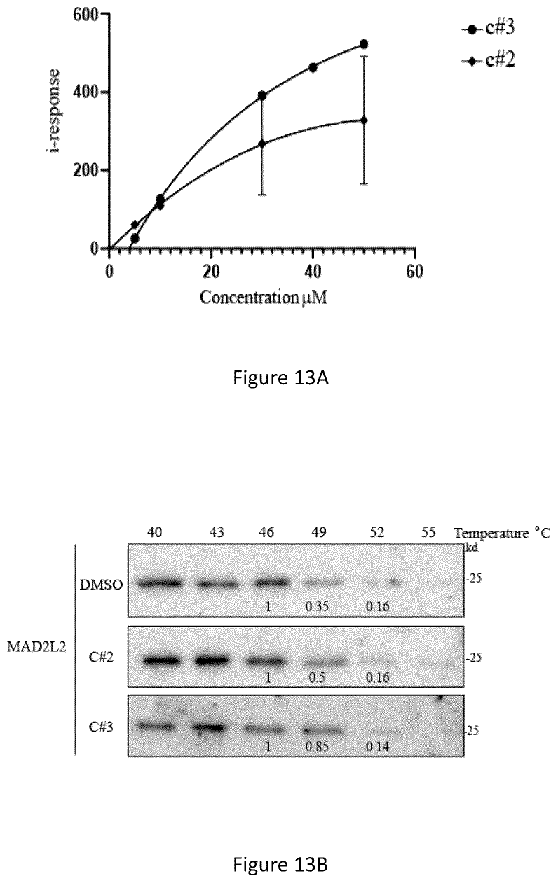

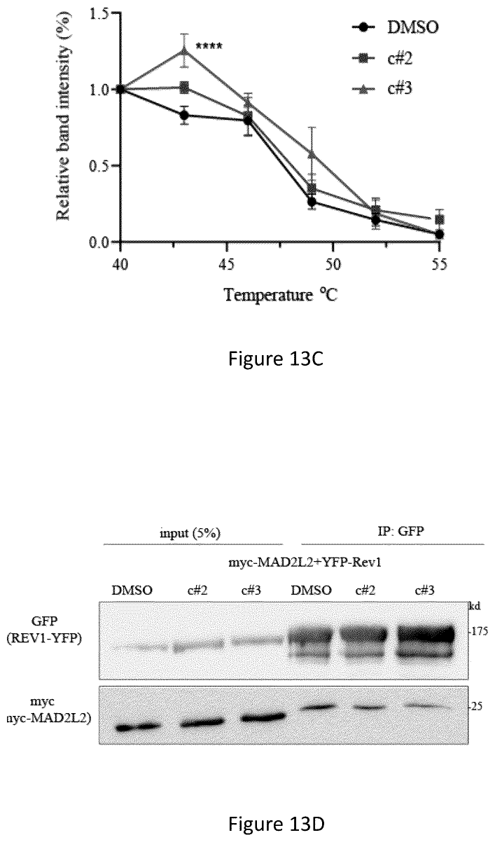

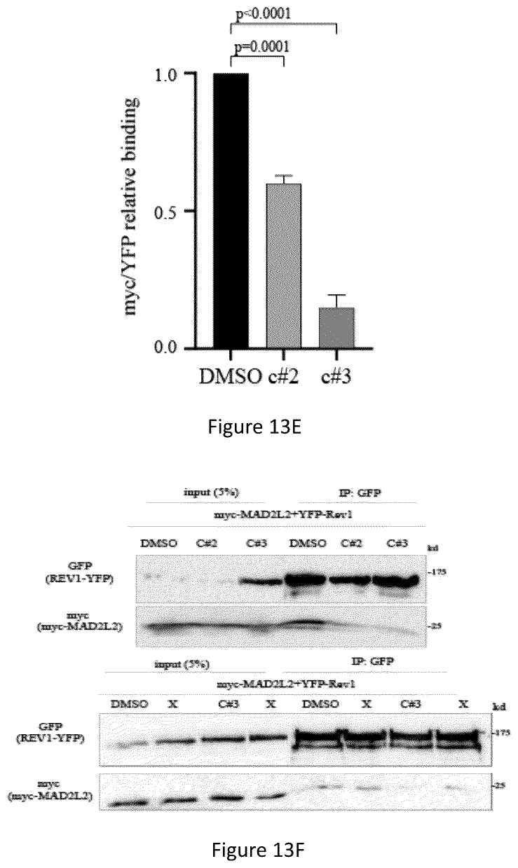

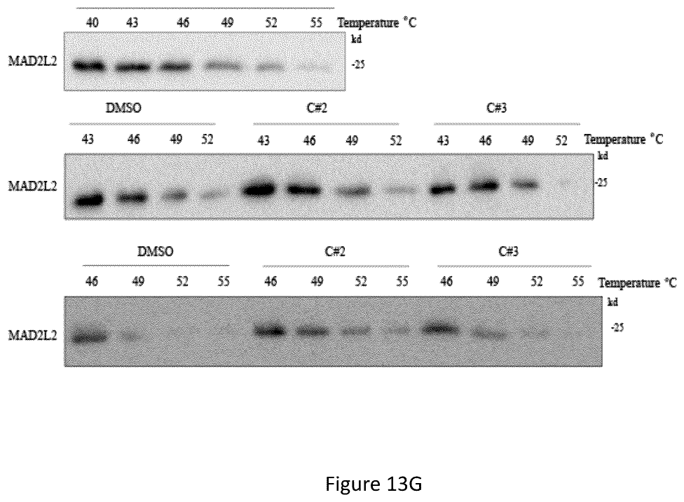

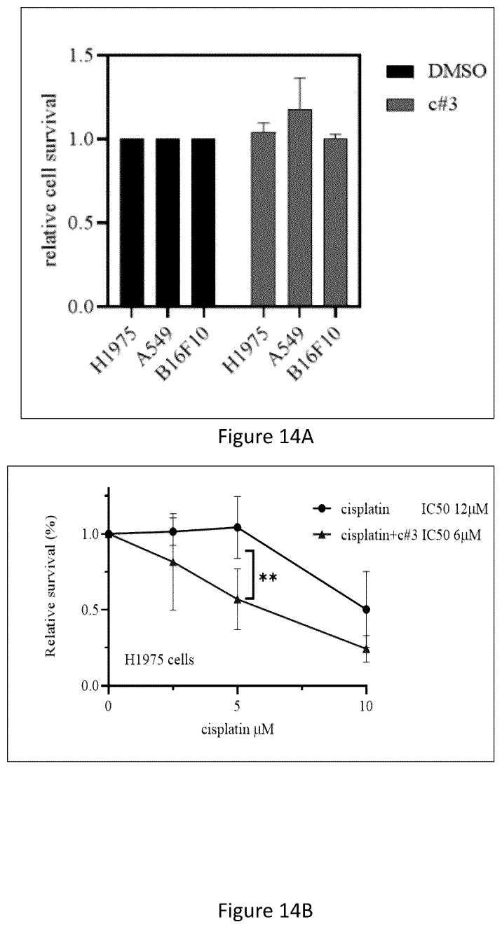

In order to validate that MAD2L2 is a direct target of c #2 and c #3, the inventors tested the compounds' binding kinetics to a recombinant protein by Field Effect Biosensing (FEB) technology. Briefly, purified GST-MAD2L2 was immobilized onto a graphite chip, and the current across the chip is measured to determine the baseline current. Then, a soluble compound was flowed over the chip. Interaction of the soluble and immobilized molecules yields a detectable change in conductance of the biosensor. The Kon and Koff of the interaction were measured over time and were used to calculate the KD. The binding of both c #2 and c #3 to MAD2L2 is in the micromolar range: 31.08 μM and 44.88 μM, respectively (FIG. 13A). The affinity of c #3 to MAD2L2 is approximately seven-fold higher in comparison to the affinity to c #2. The in vivo binding of c #2 or c #3 to MAD2L2 was validated by using a cellular thermal shift assay (CETSA), which is a reliable reporter showing whether a molecule enters the cell and the nucleus and binds to the protein of interest. Briefly, under normal conditions, most of the MAD2L2 protein unfolds, aggregates and disappears from the soluble fraction of the lysate between 46° C. and 49° C. A change in the aggregation temperature is expected upon direct binding of a compound, as the compound will affect the relative stability of the folded protein and either promote or delay its unfolding and subsequent aggregation. In this assay, we show that both c #2 and c #3 stabilize the folded MAD2L2 protein through direct binding, raising the aggregation temperature. FIG. 13C presents the temperature-induced aggregation generated after 1 hour of compound treatment and exposure of cells to a temperature gradient from 40°−55° C. Under normal conditions, MAD2L2 aggregates in the shift between 46° C. and 49° C. However, when c #3 is added, MAD2L2 remains soluble until higher temperatures, and the aggregation occurs between 49° C. and 52° C. (FIGS. 13B-C). These data suggest that c #3 binds MAD2L2 and changes its solubility. C#2 caused a milder change in MAD2L2's aggregation temperature, suggesting a weaker binding to MAD2L2.

In order to determine whether both compounds interfere with MAD2L2-Rev1 complex formation in vivo, the inventors performed co-immunoprecipitation (co-IP) of the complex. HEK293 cells were co-transfected with YFP-Rev1, together with myc-MAD2L2. Transfected cells were treated with either DMSO or with the compounds for 24h before the MAD2L2-Rev1 complex was IP'ed, using GFP-Trap beads, which recognize YFP. While YFP-Rev1 presented a clear binding to myc-MAD2L2 when no compound was added, after 24 h exposure to c #2 or c #3, a clear reduction was observed in MAD2L2-Rev1 binding (FIG. 13D), although a difference between c #2 and c #3 was observed: while c #2 reduced complex formation by about 50%, c #3 was extremely effective and reduced about 80% of MAD2L2-Rev1 binding (FIG. 13E). The biochemical data support the idea that c #3 binds better to MAD2L2 than c #2 and is probably more efficient in disrupting the MAD2L2-Rev1 complex in vivo, hence these compounds' biological effect is due to their direct binding to MAD2L2.

In this study, the inventors discovered two small molecules that potentially disturb the assembly of MAD2L2-Rev1 and the formation of an active TLS complex. Using molecular dynamics simulation, the inventors generated a new model of the MAD2L2 homodimer together with one Rev1 protein. This model exposed a unique cavity formed upon MAD2L2 homodimerization, which could serve as a new binding interface for small molecules. Hence, the MAD2L2 homodimer model was applied to a docking simulation to identify small molecules that potentially bind in this unique cavity. We performed an unbiased bioinformatics screen of approximately 5 million molecules that are available in the ZINC12 subset library. Two small molecules, c #2 and c #3, were selected for further analysis. Both compounds have lead structure, their MW<=350 and the predicted octanol-water partition coefficient XlogP[21]<=3.5. Thus, both serve as a good starting point of lead optimization in order to improve their potency, selectivity, and pharmacokinetic parameters. Hence, our approach demonstrates the importance of high-resolution structures of protein complexes that can be used for in-silico drug design.

The inventors observed that both c #2 and c #3 sensitized lung cancer cell lines to cisplatin and doxorubicin, resulting in significant cell death. A clear reduction in the measured IC50 of cisplatin and doxorubicin was observed. However, A549 cells were less sensitive to the compounds, as IC50 after combined treatment was reduced by about one third; while in the H1975 cells, combined treatment reduced IC50 by half (FIGS. 11A-C). C#2 performed slightly better when co-treated with cisplatin. The source of the sensitivity difference between cell lines is not determined. However, aneuploidy is common in many laboratory cell lines, and these genetic differences may inspire different sensitivities.

MAD2L2 is important for DNA damage tolerance and repair. Therefore, we hypothesized that the hypersensitization of the cells to cisplatin in the presence of c #2 and c #3 is the result of persistent cisplatin-induced DNA damage. Indeed, both molecules caused an increase in DNA damage when combined with cisplatin. Moreover, combined treatment caused a significant increase in the number of cells with more than 80 γH2AX foci/cell and extremely high signal, indicating that these cells suffer from severe levels of DNA damage. Interestingly, this effect was more predominant in c #3 than c #2, as c #2 increased the highly damaged cells only in A549 cells. This might be due to the higher cisplatin concentration used in the assay for A549 cells (10 μM instead of 5 μM cisplatin), as A549 cells presented intrinsic resistance to the combined treatment.

The binding of c #2 and c #3 to MAD2L2 has been confirmed in vitro. We determined the KD of the compounds to be in the micromolar range. Interestingly, the affinity of c #3 to MAD2L2 is sevenfold higher than c #2. Furthermore, the R squared value of c #2 is lower than c #3 suggesting that its binding to MAD2L2 is less stable. Next, we performed in vivo CETSA to assess the binding affinity of c #2 and c #3 in cells. In agreement with our in vitro measurements, c #3 presented a stronger binding affinity to MAD2L2 than c #2. Finally, we explored whether the biochemical interaction affects the MAD2L2-Rev1 binding. Both molecules interfere with MAD2L2-Rev1 binding; however, as expected from the measured affinities, c #3 is more potent than c #2 in preventing complex formation. Interestingly, despite the superior binding of c #3 to MAD2L2, no significant difference was observed between the molecules in cell sensitization. One potential explanation might be that c #2 may allow the formation of a partially inactive complex which can still bind to DNA, preventing or slowing TLS synthesis and potentially slowing the turnover of TLS proteins. This might explain the reduction of highly damaged cells when exposed to low cisplatin concentration, as TLS might be still functioning and prevent their appearance. C#3 efficiently prevents MAD2L2-Rev1 formation and fully prevents TLS activity, causing highly damaged cells even at a relatively low cisplatin concentration.

### Example 7

### c #3 Sensitized Cancer Cell Lines

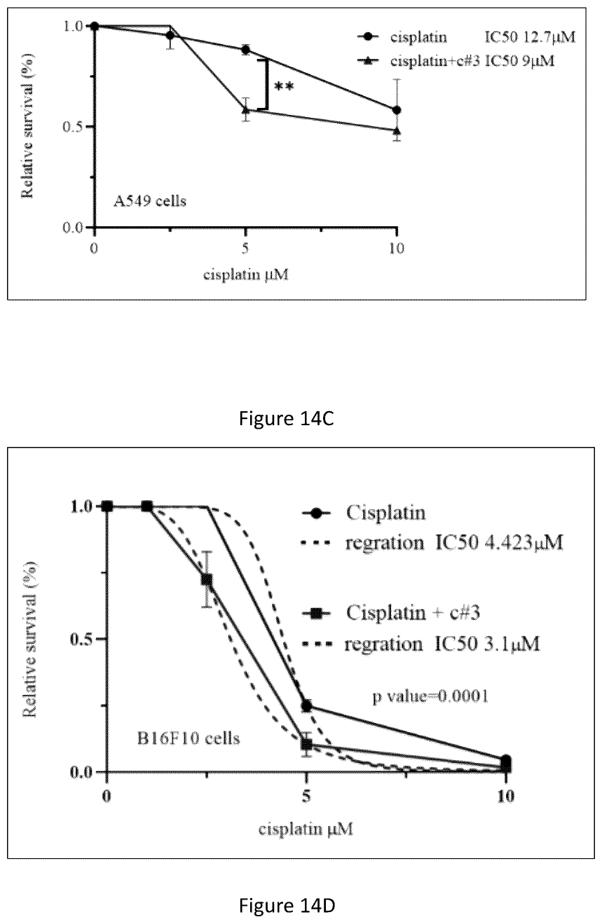

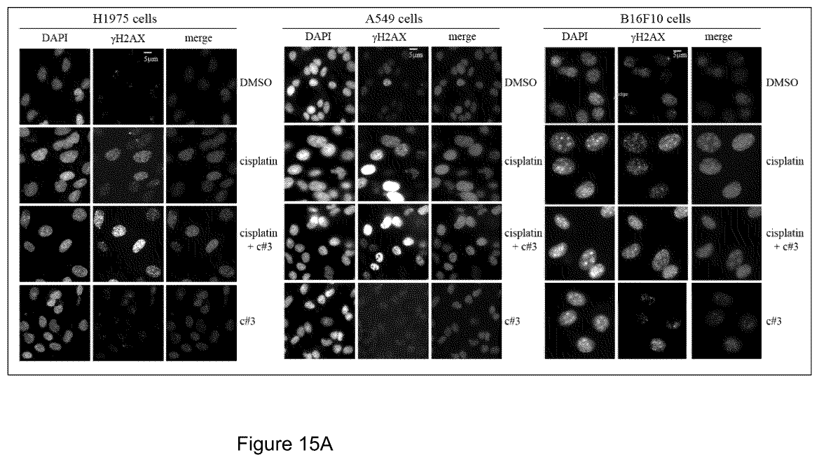

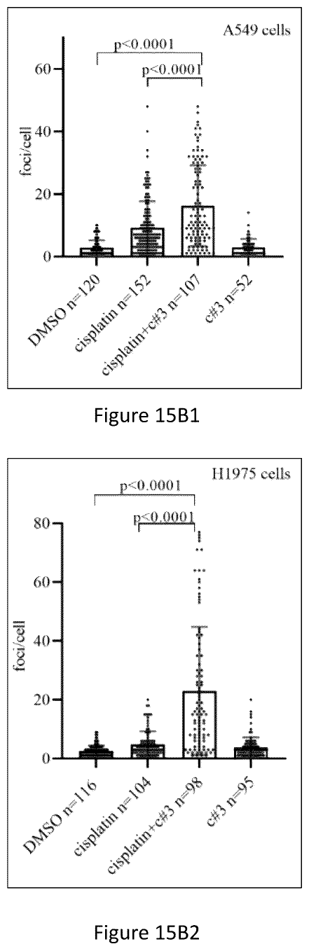

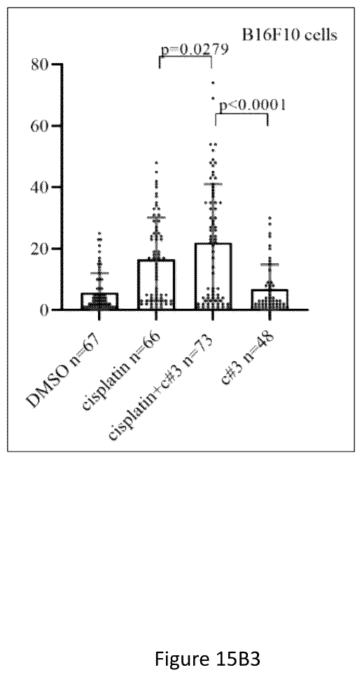

Colony survival assay to compare the relative survival rate of cells treated with cisplatin alone, or with combined treatment of cisplatin and 50 μM of compound 3 (see FIG. 1). Molecule presented preliminary promising results, We tested compound 3 (c #3) on different cancer cell lines: two lung cancer cell lines, (1) human non-small cell lung carcinoma H1975 cells, (2) epithelial lung adenocarcinoma A549 cells, and (3) mouse aggressive melanoma B16F10 cells. Notably, treatment with c #3 alone caused no toxicity during the colony survival assay (FIG. 14A).

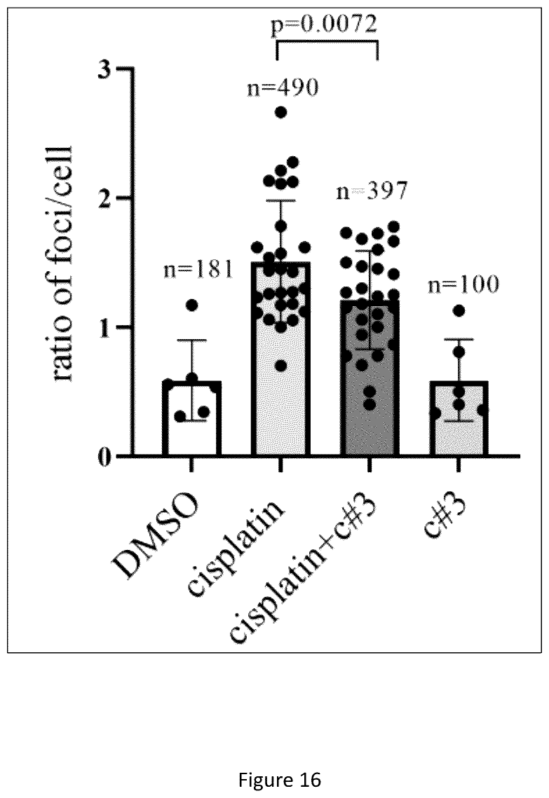

Combined treatment of cisplatin with c #3 correlates with an increase in DNA damage. Levels of γ-H2AX foci, a marker for DNA double-strand breaks, were compared in H1975, A549 and B16F10 cell lines. After 16 h treatment of cisplatin or combined treatment, a 2-fold increase in γ-H2AX foci/cell was detected in the co-treated cells (FIG. 16A, B). c #3 treatment alone had no effect on the γ-H2AX signal, and no cell death was observed. These results suggest that combined treatment indeed causes an increase in DNA damage, which correlates with enhanced cell death

#3 disturbs MAD2L2-Rev1 complex formation. Detection of MAD2L2-Rev1 binding using the proximity ligation assay (PLA), which allows endogenous detection of protein-protein interactions. To enrich for MAD2L2-Rev1 foci, cells were co-treated with 10 μM cisplatin and 100 μM c #3 for 24h. Next, the PLA reaction was performed and foci, representing the MAD2L2-Rev1 complex, were counted. Cells that were co-treated with cisplatin and c #3 presented a reduced ratio of foci per cell, compared to cells treated with cisplatin alone (FIG. 16). DMSO or c #3 alone presented similar ratios, suggesting that c #3 has no effect on complex formation when DNA-damage is not induced. Our data suggests that the reduced ratio of foci per cell in the co-treated cells reflects the binding of c #3 to MAD2L2 which prevents Rev1 binding.

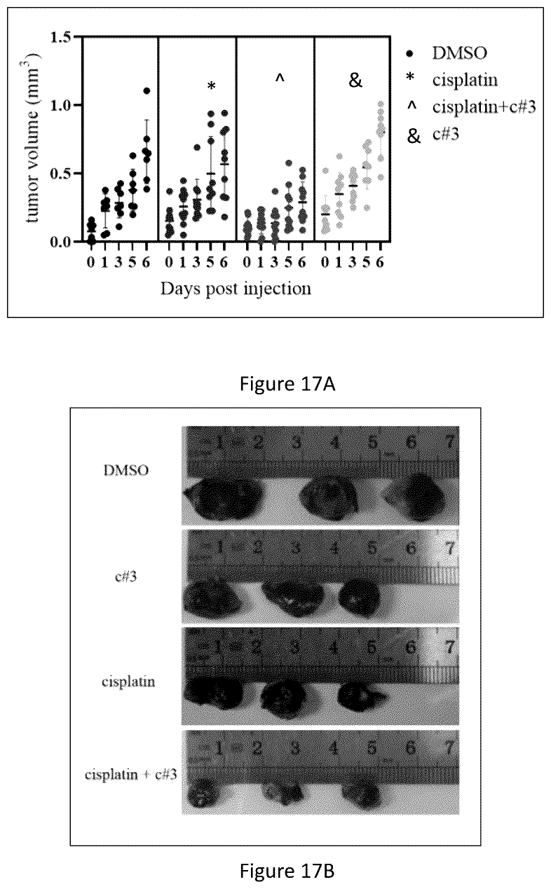

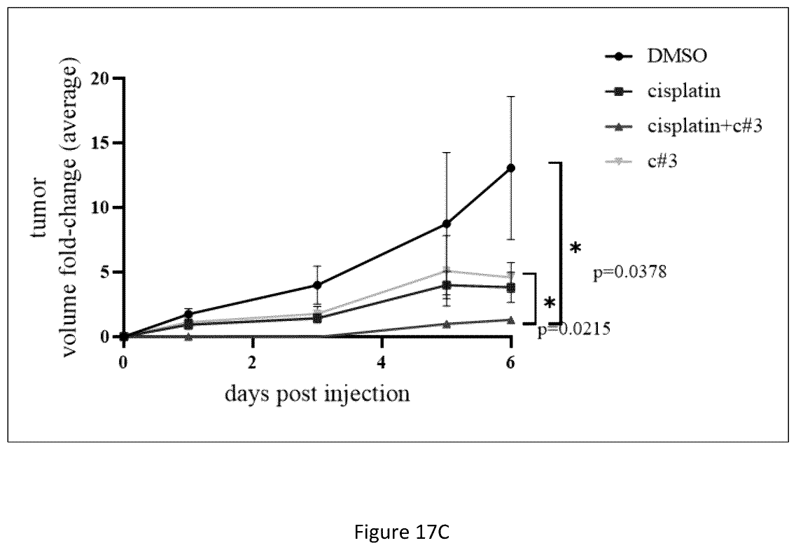

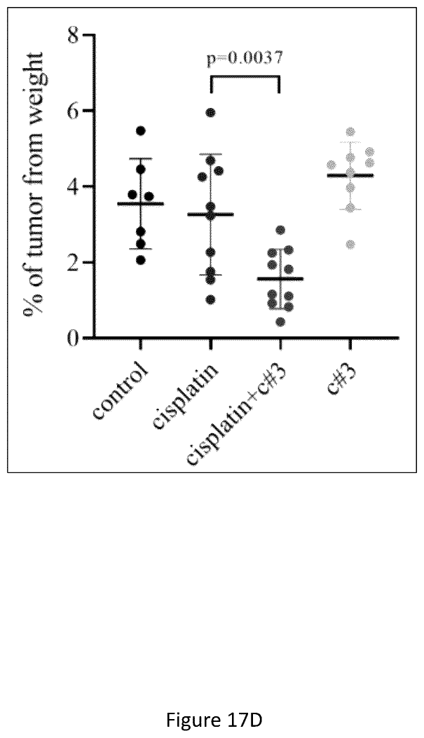

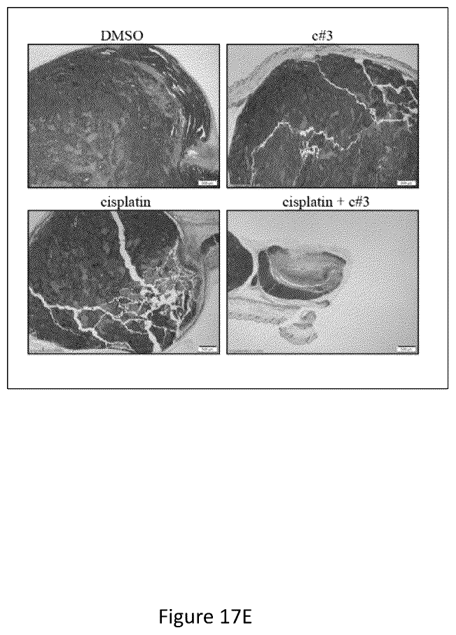

C#3 prevents tumor growth. To test the effect of c #3 in-vivo, we used a synergistic mouse model of C57BL mice. B16F10 cells were injected to the mouse's left flank and allowed to form a subcutaneous tumor. Next, we injected directly to the tumor DMSO, cisplatin, cisplatin+c #3 1 mg/kg, or c #3 alone, and tumor size was measured each day, up to 6 days. Notably, cisplatin was given at a sub-therapeutic concentration of 1 mg/kg instead of 4 mg/kg, to be able to observe the effect of the compound on tumor growth before cisplatin eliminates it. Here, the combined treatment prevented tumor growth in the first three days, where no change was observed in either total tumor volume or tumor volume fold-change (FIG. 17 A-C). Tumors that were treated with cisplatin or c #3 presented, as previously reported, similar growth inhibition. DNA damage burden in these fast-growing tumors is high, and TLS inhibition using c #3 prevents repair and enhances cell death. However, tumor co-treatment presents a significant growth inhibition when compared to all treatments. This suggests that the DNA damage caused by cisplatin is not bypassed and repaired, as TLS is inhibited, and therefore cells cannot survive. A histopathological analysis was performed on two tumors from each group, and severe necrosis was observed only in the co-treated cells. Notably, in the co-treatment, tumors started to grow three days post injection, suggesting that the compounds were circulated out of the tumor or lost activity. In addition, the average mouse weight was relatively similar in all treatment groups (not presented); however, in the co-treated group, the percentage of the tumor weight from the total mouse weight was the smallest (FIG. 17D), implying that in all control groups mice lost body weight and gained weight from the tumor. Interestingly, histopathology report, performed by PATHO-LOGICA laboratories, found that severe necrosis was observed only in the co-treated mice (FIG. 17E), this may explain the small size tumors isolated from the co-treated tumors.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A pharmaceutical composition comprising a compound, pharmaceutically acceptable salt, isomer or tautomer thereof, wherein said compound is represented by Formula II: 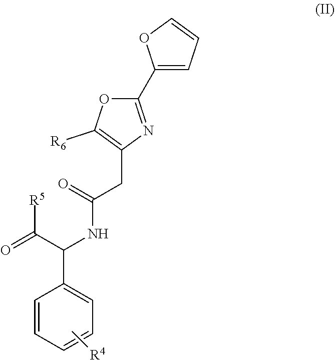
wherein:
R⁴, R⁵ and R⁶ are independently selected from the group consisting of a heteroaryl, an aryl, a bicyclic aromatic ring, an aliphatic ring, an unsaturated aliphatic ring, a bicyclic aliphatic ring, a linear C₁-C₇ alkyl group, a branched C₁-C₇ alkyl group, a branched C₁-C₇ haloalkyl group, a linear C₁-C₇ haloalkyl group, a C₁-C₇ alkylhydroxy group, a C₁-C₇ alkoxy group, a hydrogen, a hydroxy group, a halo group, an alkyl group, an alkoxy group, an amino group, or any combination thereof;
or wherein said compound is represented by Formula I: 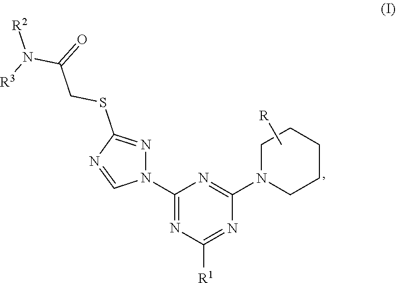
wherein:
R, R¹, R² and R³ are independently selected from the group consisting of an optionally substituted heteroaryl, an optionally substituted aryl, a optionally substituted bicyclic aromatic ring, an optionally substituted aliphatic ring, an optionally substituted unsaturated aliphatic ring, an optionally substituted bicyclic aliphatic ring, an optionally substituted linear C1-C7 alkyl group, an optionally substituted branched C1-C7 alkyl group, an optionally substituted branched C1-C7 haloalkyl group, an optionally substituted linear C1-C7 haloalkyl group, an optionally substituted C1-C7 alkylhydroxy group, an optionally substituted C1-C7 alkoxy group, a hydrogen, a heteroatom, a hydroxy group, a halo group, an alkyl group, an alkoxy group, an amino group, or any combination thereof;
or wherein said compound is represented by Formula III: 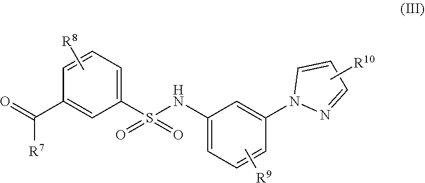
wherein:
R⁷, R⁸, R⁹ and R¹⁰ are independently selected from the group consisting of a heteroaryl, an aryl, a bicyclic aromatic ring, an aliphatic ring, an unsaturated aliphatic ring, a bicyclic aliphatic ring, a linear C₁-C₇ alkyl group, a branched C₁-C₇ alkyl group, a branched C₁-C₇ haloalkyl group, a linear C₁-C₇ haloalkyl group, a C₁-C₇ alkylhydroxy group, a C₁-C₇ alkoxy group, a hydrogen, a hydroxy group, a halo group, an alkyl group, an alkoxy group, an amino group, or any combination thereof.

2. (canceled)

3. The pharmaceutical composition of claim 1, wherein said compound is represented by Formula I and wherein R¹ represents a substituent comprising any one of —NO₂, —CN, —OH, —NH₂, carbonyl, —CONH₂, —CONR₂, —CNNR₂, —CSNR₂, —CONH—OH, —CONH—NH₂, —NHCOR, —NHCSR, —NHCNR, —NC(═O)R, —NC(═O)OR, —NC(═O)NR, —NC(═S)OR, —NC(═S)NR, —SO₂R, —SOR, —SR, —SO₂OR, —SO₂N(R)₂, —NHNR₂, —NNR, C₁-C₇ haloalkyl, optionally substituted C₁-C₁₀ alkyl, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), amino(C₁-C₆ alkyl), —CONH(C₁-C₆ alkyl), —CON(C₁-C₆ alkyl)₂, —CO₂H, —CO₂R, —OCOR, —C(═O)R, —OC(═O)OR, —OC(═O)NR, —OC(═S)OR, —OC(═S)NR, a heteroatom, an optionally substituted cycloalkyl, an optionally substituted heterocyclyl, an optionally substituted aryl, or a combination thereof.

4. (canceled)

5. The pharmaceutical composition of claim 1, wherein said compound is selected from:

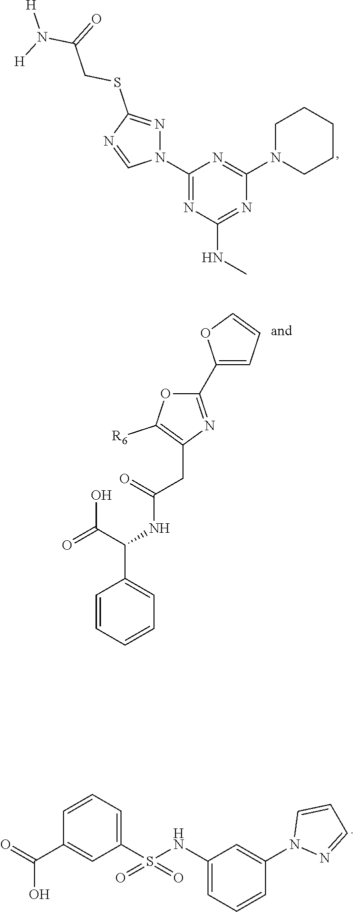

6.-15. (canceled)

16. A method for treating or preventing development of cancer in a subject in need thereof, comprising administering to said subject a therapeutically effective amount of (a) chemotherapeutic agent and (b) the pharmaceutical composition of claim 1.

17. The method of claim 16, wherein said cancer is selected from the group consisting of lung cancer, breast cancer, osteosarcoma, neuroblastoma, colon adenocarcinoma, chronic myelogenous leukemia (CML), acute myeloid leukemia (AML), acute promyelocyte leukemia (APL), sarcoma, myxoma, rhabdomyoma, fibroma, lipoma, teratoma; bronchogenic carcinoma, alveolar carcinoma, bronchial adenoma, sarcoma, lymphoma, chondromatous hamartoma, mesothelioma, esophageal cancer, stomach cancer, pancreatic cancer, small bowel cancer, large bowel cancer; kidney cancer, bladder cancer, urethra cancer, prostate cancer, testis cancer; hepatoma, cholangiocarcinoma, hepatoblastoma, angiosarcoma, hepatocellular adenoma, hemangioma, osteogenic sarcoma, fibrosarcoma, malignant fibrous histiocytoma, chondrosarcoma, Ewing's sarcoma, malignant lymphoma, multiple myeloma, malignant giant cell tumor chordoma, osteochronfroma, benign chondroma, chondroblastoma, chondromyxofibroma, osteoid osteoma, giant cell tumors, cancer of the skuli, meninges cancer, brain cancer, spinal cord cancer, uterus cancer, cervical cancer, cancer of the ovaries, vulva cancer, vagina cancer, Hodgkin's disease, non-Hodgkin's lymphoma, malignant melanoma, basal cell carcinoma, squamous cell carcinoma, Karposi's sarcoma, moles dysplastic nevi, lipoma, angioma, and dermatofibroma.

18. (canceled)

19. The pharmaceutical composition of claim 1, further comprising a chemotherapeutic agent.

20. The pharmaceutical composition of claim 19, comprising instructions for administering said pharmaceutical composition and said chemotherapeutic agent a w/w ratio ranging from 1:0.01 to 0.01:1.

21.-38. (canceled)

39. A method for determining suitability of a compound to (i) treating or preventing development of cancer and/or (ii) increasing or prolonging the therapeutic efficacy of a chemotherapeutic agent, the method comprising contacting the compound with a pocket domain within a region of a Mitotic Arrest Deficient 2 Like 2 protein (MAD2L2; SEQ ID NO: 1), wherein binding of the compound to said pocket is indicative of the compound being effective in (i) treating or preventing development of cancer and/or (ii) increasing or prolonging the therapeutic efficacy of a chemotherapeutic agent.

40. The method of claim 39, wherein the binding is to one or more of: SEQ ID NO: 2 (LLAAFILK); and SEQ ID NO: 3 (LIPLKTMTSDILKMQLYV).

41. The method of claim 39, wherein the binding is determined by inhibition of MAD2L2:Rev1 interaction.
